(19) United States
(12) Patent Application Publication
(10) Pub. No.: US 2023/0258633 A1
(43) Pub. Date: Aug. 17, 2023

(54) METHOD FOR ANTIGEN DETECTION FROM DIRECT CLINICAL SAMPLES

(71) Applicant: The Regents of the University of California, Oakland, CA (US)

(72) Inventors: Timothy RODWELL, La Jolla, CA (US); Antonino CATANZARO, La Jolla, CA (US); Marva SEIFERT, La Jolla, CA (US); Joseph WANG, La Jolla, CA (US); Eva Vargas ORGAZ, La Jolla, CA (US); Victor Ruiz Valdepenas MONTIEL, La Jolla, CA (US)

(73) Assignee: The Regents of the University of California, Oakland, CA (US)

(21) Appl. No.: 18/014,220

(22) PCT Filed: Jul. 7, 2021
(86) PCT No.: PCT/US21/40672

§ 371 Date: Jan. 3, 2023

Related U.S. Application Data
(60) Provisional application No. 63/048,989, filed on Jul. 7, 2020.
(60) Provisional application No. 63/067,963, filed on Aug. 20, 2020.

(51) Int. Cl. G01N 33/569; G01N 27/327; G01N 33/553; C07K 16/12; C07K 16/10

(52) U.S. Cl. CPC G01N 33/569; G01N 27/327; G01N 33/553; C07K 16/1289; C07K 16/10; G01N 2333/35; G01N 2333/165

(57) ABSTRACT

In one aspect, the disclosure relates to test strips for detecting and/or monitoring the treatment of a disease in a subject. The test strips are inexpensive and disposable and can be used directly with clinical biological samples from patients including, but not limited to, blood, plasma, saliva, and urine. Also disclosed are methods of using the test strips to quantify antigens produced by a virus or microorganism causing an infectious disease. The methods can be conducted at a point of care for a patient and do not require expensive equipment or extensive operator training. The methods are also rapid to complete and can be used to monitor progress in the treatment of a disease This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/048,989 filed on Jul. 7, 2020, and 63/067,963 filed on Aug. 20, 2020, each of which is incorporated herein by reference in its entirety.

### STATEMENT REGARDING FEDERALLY SPONSORED RESEARCH OR DEVELOPMENT

This invention was made with government support under AI036214-25 awarded by the National Institutes of Health. The government has certain rights in the invention.

### FIELD OF INVENTION

The present disclosure relates generally to a diagnostic method, particularly for detection of SARS-CoV-2 and tuberculosis antigens.

### BACKGROUND OF THE INVENTION

Rapid and inexpensive testing for respiratory and other infections is critical for identifying infectious individuals, implementing isolation and/or quarantine protocols, and halting the spread of infectious diseases, as well as monitoring the progress of treatment in patients, especially patients infected with drug-resistant pathogens.

The global reference standard-of-care for diagnosing tuberculosis (TB) in patients at risk for disease is to culture Mycobacterium tuberculosis (Mtb) from patient sputum samples, which takes several weeks, requires expensive and complex biosafety facilities to complete, and is very difficult to accomplish when patients such as those living with HIV and children, cannot produce sufficient Mtb in their sputum for culture. There are currently no US Food and Drug Administration (FDA) approved or World Health Organization (WHO) endorsed antigen detection diagnostic assays for detection of active TB disease using blood samples. There is currently no FDA approved or WHO endorsed assay for diagnosing TB at the point of care (POC). There is only one FDA approved molecular test for detection of TB from sputum (Cepheid GeneXpert). It is considered a moderately complex test that is at best “near-patient”, not true POC, and costs up to several hundred dollars per test in the US due to the high cost of goods and complexity of the assay. There is a urine-based LAM antigen detection assay endorsed by WHO for TB diagnosis—but it is only endorsed for use in a very select group of HIV patients with a certain CD4 level due to its poor performance outside of this narrowly defined patient population.

The SARS-CoV-2 (CV2) virus has now infected approximately 180 million people and caused almost 4 million deaths globally, making it one of the deadliest pandemics in recent history. With no effective therapeutics, uneven global vaccine distribution, differences in effectiveness of vaccines, age-variable mortality, and the emergence of new, more transmissible strains, as well as with up to 40% of infected individuals presenting with high viral loads but no symptoms, it is nearly impossible to return to pre-pandemic normal activities without risking morbidity and mortality in the most vulnerable, including in individuals too young to receive currently-approved vaccinations and immunocompromised patients. In such an environment, one important way to lower individual and population risks is to test individuals for COVID-19 broadly and frequently. In the US, with total cases topping 50,000/day during some periods of 2020, COVID-19 testing capacity has been hovering well below what experts estimate is an “adequate” level of testing, which ranges from 900,000 to 20 million tests/day. In low and middle income countries (LMIC), it is also estimated that over 500 million COVID tests will be needed in the next 12 months. The vast majority of the diagnostic burden in the US and world is still being carried by centralized laboratories using high throughput real-time PCR (RT PCR) for detection of CV2 viral RNA from processed clinical samples, collected with nasopharyngeal (NP) and oropharyngeal (OP) swabs. While several RT PCR platforms have recently been approved for near-patient and point-of-care (POC) COVID-19 diagnosis, all RT PCR solutions are limited by extremely high cost (>$100/sample), complex manufacturing requirements (limiting supply of instruments and cartridges) and supply chain shortages of swabs, viral transport medium, and PCR reagents. While RT PCR will always be a necessary component of the US and global testing strategy, this approach is not sufficient to meet the massive global demand for cheap, rapid, decentralized serial clinical testing, and it is anticipated that unless the COVID-19 diagnostic strategy is fundamentally transformed, testing will continue to lag demand, leaving transmission risks unchecked.

While SARS-CoV-2 has temporarily displaced Mycobacterium tuberculosis (Mtb) as the leading cause of infectious disease mortality worldwide, it is expected that the COVID-19 pandemic might ultimately add 400,000 excess tuberculosis (TB) deaths to the annual TB mortality of 1.4 million TB deaths reported in 2019. The World Health Organization (WHO) estimates that approximately 10 million individuals develop active TB annually, but 30% of these (3 million) are never diagnosed or reported. This gap in diagnosis is due in part to the lack of a rapid, highly sensitive, and inexpensive point-of-care TB diagnostic solution that can be deployed at the lowest levels of the healthcare system. Treatment delays due to these gaps in diagnosis are linked to poor treatment outcomes and contribute to continued transmission. Furthermore, as new infectious diseases emerge, many of which may have overlapping symptoms, accuracy of diagnosis will ensure selection of appropriate treatment at an earlier stage of illness, improving patient outcomes and reducing transmission.

Antigen tests are characterized by: 1) simple manufacturing requirements—suitable for rapid and low cost scaling; 2) low complexity workflows adaptable for direct clinical sample processing and non-expert handling; 3) low cost of goods (COGs)<$5 per test; and 4) excellent specificity (˜100%) due to high affinity antibody/antigen bonding. To date, however, the main drawback of CV2 antigen assays has been low sensitivity, such that both the US Food and Drug Administration (FDA) and World Health Organization (WHO) have preliminary target product profiles (TPPs) to guide developers that only require CV2 antigen assays to achieve 80% positive percent agreement with reference testing. In low to moderate CV2 prevalence environments, such as most of the US, this means CV2 antigen tests with these specifications would have such low negative predictive value, that they would still require additional RT PCR testing after a negative antigen test result, with the same capacity limitations currently being experienced. Currently, only two CV2 antigen detection assays are approved by the FDA under Emergency Use Authorization (EUA), the Veritor Plus™ (BD, USA) and the Sofia SARS Antigen Fluorescent Immunoassay (Quidel, USA). Both assays require nasal swab sample collection and have a published sensitivity of <85%.

Despite advances in rapid testing for infectious diseases including, but not limited to, tuberculosis and COVID-19, there is still a scarcity of methods and apparatuses that are sensitive, selective, and rapid, while also being able to be produced for low cost and used by operators requiring minimal specialized training. These needs and other needs are satisfied by the present disclosure.

### SUMMARY OF THE INVENTION

In accordance with the purpose(s) of the present disclosure, as embodied and broadly described herein, the disclosure, in one aspect, relates a test strip for detecting a disease or monitoring treatment of a disease in a subject, the test strip comprising a working electrode and a combination reference and counter electrode, wherein the working electrode and the combination reference and counter electrode comprise a substrate coated with a thin film of a metal such as gold, and wherein the metal is bonded to a detection antibody for an antigen associated with the disease by a method such as, for example, carboxyl-amine coupling, a thiolate self-assembled monolayer, 4-carboxymethylaniline conjugation, or another method. In one aspect, the substrate comprises a plastic material such as, for example, polyethylene terephthalate glycol (PETG).

In another embodiment the disease can be an infectious disease caused by a virus such as, for example, severe acute respiratory syndrome virus 2 (SARS-CoV-2), poxvirus, human papillomavirus, parvovirus, lassa virus, rotavirus, herpes simplex virus types 1 or 2, influenza virus, human immunodeficiency virus (HIV), human T cell leukemia virus (HTLV), Epstein-Barr virus (EBV), human cytomegalovirus (HCMV), Kaposi's sarcoma-associated herpesvirus (KSHV), varicella-zoster virus (VZV), hepatitis A virus, hepatitis B virus, hepatitis C virus, hepatitis D virus, hepatitis E virus, Ebola virus, Marburg virus, parainfluenza virus, human respiratory syncitial virus, Hendra virus, Nipah virus, mumps virus, measles virus, hantavirus, bunyavirus, Rift Valley fever virus, sin nombre virus, rabies virus, an encephalitis virus, West Nile virus, yellow fever virus, Dengue virus, norovirus, rubella virus, Zika virus, severe acute respiratory syndrome virus (SARS-CoV), Middle East respiratory syndrome (MERS), or another coronavirus.

In a further embodiment the disease can be an infectious disease caused by a bacterium such as, for example, Bacillus anthracis, Bacillus cereus, Bartonella henselae, Bartonella quintana, Bordetella pertussis, Borrelia burgdorferi, Borellia garinii, Borrelia afzelii, Borellia recurrentis, Brucella abortus, Brucella canis, Brucella melitensis, Brucella suis, Campylobacter jejuni, Chlamydia pneumonia, Chlamydia trachomatis, Chlamodyphila psittaci, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis (tuberculosis), Mycobacterium ulcerans, Mycoplasma pneumonia, Neisseria gonorrhoeae, Neisseria meningitides, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Ureaplasma urealyticum, Vibrio cholera, Yersinia pestis, Yersinia enterocolitica, or Yersinia pseudotuberculosis.

In still another embodiment the disease can be an infectious disease caused by a fungus such as, for example, Aspergillus fumigatus, Aspergillus niger, Blastomyces dermatitidis, Candida albicans, Candida auris, Candida glabrata, Candida parapsilosis, Coccidiodes immitis (e.g. coccidiomycosis), Coccidioides posadasii (e.g. coccidiomycosis), Cryptococcus neoformans, Cryptococcus gattii, Epidermophyton floccosum, Trichophyton interdigitale, Trichophyton mentagrophytes, Trichophyton rubrum, Trichophyton tonsurans, Histoplasma capsulatum, Rhizopus oryzae, Pneumocystis jirovecii, Sporotrichosis schenckii, or Sporothrix brasiliensis.

In any of these embodiments, the capture antibody can be selected to bind to one or more antigens related to the disease organism. In another aspect, the capture antibody can have a capture efficiency of at least 50% and can be stable on the substrate (i.e., can retain at least 90% of an initial activity level) after storage at low temperature (e.g., 4° C.) for up to a month. In another aspect, the test strips are inexpensive and disposable.

Also disclosed are methods for detecting disease in the subject comprising at least the steps of incubating the disclosed test strips with a biological sample (e.g., blood, serum, urine, or saliva) from the subject, rinsing the test strips, incubating the test strip with a detection antibody specific to the at least one disease-related antigen, wherein the detection antibody comprises at least one tag, rinsing the test strip a second time to remove unbound detection antibody, and detecting a signal from the at least one tag. In one aspect, the tag can be an enzyme label or can include horseradish peroxidase (HRP). In a further aspect, when the tag comprises HRP, the test strip can be contacted with a solution of 3,3′,5,5′-tetramethylbenzidine (TMB) and hydrogen peroxide followed by application of a current to the test strip in order to generate an amperometric signal, which can be measured using a potentiostat or potentiometer. In some embodiments, the potentiostat or potentiometer can be a mobile device. In an aspect, the biological sample can be used to contact the test strip directly, without the need for any additional sample preparation steps.

In one aspect, the amperometric signal can be from about 30 nA to greater than about 100 nA. In a further aspect, when the signal is about 30 nA, the biological sample can be free antibodies to the infectious disease (i.e., the subject does not have the disease). In another aspect, when the signal is about 100, 110, 120, 130 nA, or greater, the biological sample includes antibodies to the infectious disease (i.e., the subject has the disease).

In any of these aspects, the magnitude of the amperometric signal corresponds to a concentration of the antigen in the biological sample and/or is directly proportional to the amount of HRP-tagged detection antibody bound to the test strip. In an alternative aspect, when no amperometric signal is generated, the subject does not have the disease.

The disclosed methods can detect antigen concentrations as low as about 100 pM, or from about 100 pM to about 8.8 nM in a 10 μL biological sample, or about 100, 200, 300, 400, 500, 600, 700, 800, or 900 pM, or about 1, 2, 3, 4, 5, 6, 7, 8, 9, or about 10 nM, or a combination of any of the foregoing values, or a range encompassing any of the foregoing values. In another aspect, the disclosed methods can detect from about 10 to about 1000 viral particles in a 10 μL biological sample, or about 10, 50, 100, 500, or about 1000 viral particles, or a combination of any of the foregoing values, or a range encompassing any of the foregoing values. In still another aspect, the disclosed methods can be completed in under about 90 minutes, or under about 90, 75, 60, 45, 30, or 20 minutes. In some aspects, the disclosed methods can be completed on-site at a point of care (POC) for the subject.

Also disclosed are methods for monitoring treatment of an infectious disease in a subject, the methods including performing the disclosed detection methods in a subject having the infectious disease a first time to obtain a first antigen quantity, treating the infectious disease, and performing the disclosed detection methods in the subject a second time to obtain a second antigen quantity, wherein when the second antigen quantity is lower than the first antigen quantity, disease treatment is successful.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show a schematic of tuberculosis antigen detection using the disclosed methods. FIG. 1A shows a schematic diagram of an exemplary chip according to one embodiment and representative chronoamperograms from TB-infected and uninfected individuals. FIG. 1B shows an image of an exemplary chip with two gold-sputtered electrodes, one employed as a working electrode (WE) and one as a reference/counter electrode (RE/CE). FIG. 1C is a schematic of proof-of-concept 2-step sandwich-type immunoassay, based on the sequential incubation of 1) the sample and capture antibody (Cab) followed by 2) the horseradish peroxidase-tagged detection antibody (HRP-DAb). In this exemplary embodiment, 45 min are used for each incubation, then the 2.5 mins for reading of the sensor using electrochemical chronoamperometry based on an H₂O₂/HRP/TMB redox reaction.

FIG. 2A is a photograph of a sheet of fabricated sensors. FIG. 2B shows a single sensor adapted for a TB assay.

FIG. 3 shows a schematic of modification of an exemplary disclosed sensor with a stable thiol self-assembled monolayer (SAM), followed by incubation with Cab and blocking with ethanolamine.

FIGS. 4A-4D show characterization of the disclosed immunostrip detection method and its performance in buffer. FIG. 4A shows electrochemical performance and characterization of the buffer optimized Bioelectronic TB (BET) assay using CFP-10 spiked into negative control buffer as sample. Chronoamperograms and calibration plot obtained from the analysis of 0.0, 10, 50, 100 and 250 nM CFP-10 standards spiked into buffer. Current intensity (i) and cathodic current intensity (ic) measured in nA. FIG. 4B shows electrochemical performance and characterization of the Bioelectronic TB (BET) sensor based on CFP-10 spiked into negative control phosphate buffer. Nyquist curves resulting from the electrochemical characterization of the sequential modification steps used to develop the BET immunoassay.

FIGS. 5A-5C show electrochemical performance and characterization of the serum and urine optimized TB assay using CFP-10 spiked into negative control serum and urine as samples. Chronoamperometric responses and calibration plots obtained from the analysis of 0.0 (*, black lines and white bars) to 25 nM (+) CFP10 standard spiked into human serum (FIG. 5A), saliva (FIG. 5B), and urine (FIG. 5C). Current intensity (i) and cathodic current intensity (ic) measured in nA.

FIGS. 6A-6C show analysis of samples from infected tuberculosis patients using the disclosed methods. An asterisk indicates the tuberculosis patient was being medicated for tuberculosis at the time of testing. The control was human serum stripped using charcoal (4×).

FIGS. 7A-7D show analysis of samples from infected tuberculosis patients using the disclosed methods.

FIGS. 8A-8J show results of optimization experiments for critical bioelectronic TB (BET) sensor and assay parameters in phosphate buffer. Chronoamperometric signals obtained from evaluated parameters of a novel BET assay using phosphate buffer as a negative control or Blank (labeled “B” and shown with white bars) and phosphate buffer spiked with 100 nM of Mtb CFP10 as a positive control or Signal (labeled “S” and shown with grey bars). Graphs show change in signal-to-blank (S/B) ratio (lines) with different parameters: FIG. 8A) MUA concentration; FIG. 8B) MCH concentration; FIG. 8C) Cab concentration; FIG. 8D) Ethanolamine concentration; FIG. 8E) HRP-DAb concentration; FIG. 8F) Number of incubation steps; FIG. 8G) HRP-DAb incubation media; FIG. 8H) CAb incubation time; FIG. 8I) antigen target incubation time; FIG. 8J) HRP-DAb incubation time. Error bars were estimated as the standard deviation of three replicates.

FIGS. 9A-9E show results of optimization experiments for critical bioelectronic TB (BET) assay parameters in serum. Chronoamperometric signals obtained from evaluated parameters of a novel BET assay using charcoal stripped (4×) human serum as a negative control or Blank (labeled “B” and shown with white bars) and charcoal stripped serum spiked with 25 nM of Mtb CFP10 as a positive control or Signal (labeled “S” and shown with grey bars). Graphs show change in signal-to-blank (S/B) ratio (lines) with different assay parameters: FIG. 9A) CAb concentration; FIG. 9B) Number of incubation steps; FIG. 9C) antigen target incubation time; FIG. 9D) HRP-DAb concentration; FIG. 9E) HRP-DAb incubation time. Error bars were estimated as the standard deviation of three replicates.

FIGS. 10A-10B show background signals observed with the Bioelectronic TB (BET) assay from negative control serum and urine matrices, and effect of blocking. FIG. 10A) Chronoamperometric responses obtained from the buffer-optimized BET immunoassay protocol compared to the responses after applying a blocking step to the sensor fabrication; using undiluted (i, ii) or diluted (iii) commercial horse serum for 30 (i, iii) or 15 minutes (ii) incubation on the sensor. Measurements were made from undiluted negative control urine samples (B—striped bars) and urine spiked with 10 nM of Mtb CFP10 antigen standard (S—solid bars). The lines above the bars and diamonds indicate S/B ratio for each positive/negative control pair. Error bars were estimated as the standard deviation of three replicates for each. FIG. 10B) Background chronoamperometric responses of the BET assay in the absence of CFP10, obtained from negative controls consisting of buffer, commercially acquired charcoal stripped (4×) human serum and urine matrices.

FIG. 11 shows matrix effects of the disclosed method in human biological fluids. See also Table 3.

FIGS. 12A-12D show construction of an exemplary device useful in the disclosed method. FIG. 12A: Sensor array mass-produced by sputtering gold on PETG substrate. FIG. 12B: Single disposable sensor. FIG. 12C: Schematic of sensor comprised of the gold working electrode (WE) showing the immobilized capture antibody for specific antigen recognition, where the detection is localized, and a second gold electrode acting as joint reference/counter electrode (RE/CE) FIG. 12D: Immunoreaction measurement at −0.1V the reduction current of the HRP catalyzed H₂O₂/TMB redox probe.

FIG. 13 shows cross reactivity studies of commercial anti-S1 and anti-S1(receptor binding domain)-Ab acquired from Sinobiological with SARS-CoV-1 (CV1), Middle East Respiratory Syndrome (MERS), and other currently circulating human coronaviruses.

FIGS. 14A-14C show CV2 electrochemical immunoassay (ECIA) amperometric responses to CV2 receptor binding domain (RBD) antigen spiked into saliva collected using (FIG. 14A) Sarstedt's Salivette or (FIG. 14B) direct collection into a tube. Top curves and left column bars indicate disease-free saliva and bottom curves and right column bars show saliva spiked with 2 nM of RBD. The signal:blank (S/B) ratio (line across columns in FIG. 14C) indicates spit sample had higher signal relative to control than salivette collected.

FIGS. 15A-15C show evaluation of the heterogeneity of saliva samples from different subjects. Chronoamperograms (FIGS. 15A-15B) and amperometric responses (FIG. 15C) obtained in the measurement of spit saliva samples collected from different subjects (i, ii) unspiked (top curves and left column bars) and spiked with 2 nM of RBD (bottom curves and right column bars). Line across columns in FIG. 15C indicates sensitivity achieved. Error bars=SD of two replicates.

FIGS. 16A-16C show chronoamperograms (FIG. 16A) and corresponding calibration plot (FIG. 16B) obtained during the ECIA assay detection of 0 (top line), 0.5 (second line from top), 1.0 (third line from top) and 2.0 (bottom line) nM of CV2 RBD antigen spiked into fresh saliva. Reproducibility (FIG. 16C) of the ECIA results for the measurement of control (top curves curves/left column bars) and 2 nM RBD (bottom curves/right column bars) spiked saliva samples (each measured with 3 sensors). Error bars=SD of two replicates.

FIG. 17 shows serial dilution calibration with a sample (SB_0013). Iglewicz and Hoaglin's robust test for multiple outliers (two-sided test): Outlier criterion: Modified Z score 3.5.

FIG. 18 shows experiments in spit saliva and comparison with conventional salivette protocol. For spit saliva, measurements were conducted using a PalmSens potentiostat; For conventional salivette protocol saliva, measurements were conducted using an Autolab potentioStat.

FIG. 19 shows calibration in undiluted spit saliva. Immunoassay conditions: Cab 100 μg/mL, 4-5 min; Ethanolamine 2M, 30 min; and 1-step assay: RBD-His antigen+HRP-Ab 3.0 μg/mL, 20 min. Measurements were conducted using a PalmSens potentiostat.

FIGS. 20A-20B show Reproducibility and stability of a bioelectronic TB (BET) assay in buffer. FIG. 20A) Reproducibility of the BET assay using both phosphate buffer negative controls×6 (a, top lines and left bars) and phosphate buffer spiked with 100 nM Mtb CFP-10 antigen×6 positive controls (b, bottom lines and right bars). FIG. 20B) Amperometric results from 3×replicate BET assay runs on each of 7 days over a 30-day period after fabrication and storage at 4° C., using buffer spiked with 100 nM CFP-10 as a positive control. Current intensity (i) and cathodic current intensity (ic) measured in nA.

FIGS. 21A-21E show amperometric response of the bioelectronic TB assay using clinical serum and urine samples from patients at risk for TB. Amperometric responses obtained from the assay using clinical serum (FIG. 21A) and urine (FIG. 21B) samples. Results from 3× replicates of negative control serum and urine (white bars), TB negative patient serum and urine (black bars), TB positive patient serum and urine with zero days of treatment before sample collection (TB—untreated: light gray bars) and TB positive patient serum and urine with 1-17 days of TB treatment before sample collection (TB—treated: dark gray bars). Black line represents tentative lower threshold for diagnosis of TB positivity. Box and whiskers plot of mean and interquartile range (IQR) of amperometric signals from clinical serum (FIG. 21C) and urine (FIG. 21D) from TB negative (circle dots), untreated TB positive patients (triangle dots) and treated TB positive patients (diamond dots). Correlation of amperometric responses obtained from paired serum and urine samples (FIG. 21E). Dots and error bars represent mean and standard deviation of three replicates. Cathodic current intensity (ic) measured in nA.

FIGS. 22A-22B show amperometric results from a bioelectronic TB assay using paired clinical serum samples from the same patient over time to indicate effect of treatment days on signal. FIG. 22A) Amperometric responses obtained from the assay using serial clinical serum samples collected from the same TB positive patients on day zero before drug resistant TB treatment (TB—untreated: dark gray bars) and after pre-set number of treatment days (TB—treated: black bars). Results from 3× replicates of negative control serum (white bar) and each clinical sample. Dashed black line represents tentative lower threshold for diagnosis of TB positivity. Numbers represent number of days of treatment. FIG. 22B) Amperometric signal strength from paired, serial serum samples from individual patients, plotted against days of treatment and stratified by culture status of the patient at follow up time point. Triangle symbols and dashed line represent patients with AFB/culture negative sputa at follow up time point and oval symbols, * and dashed line represent patients with AFB negative/Culture positive sputa at follow up. Cathodic current intensity (ic) measured in nA.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

In accordance with the purpose(s) of the present disclosure, as embodied and broadly described herein, the disclosure, in one aspect, relates to a POC antigen detection assay with a performance that matches culture-based reference standards and that is also low-cost, rapid, and has the potential to work on almost any clinical sample. The assay is simple to fabricate and has the potential for massive, low-cost scaling worldwide, making diagnosis of TB and other respiratory diseases, including, but not limited to, COVID-19 as well as diseases caused by emerging pathogens, much simpler and faster on a global scale.

In certain embodiments, the present disclosure provides a novel point-of-care (POC), rapid electrochemical immunoassay (ECIA) capable of detecting clinically-relevant, ultra-low concentrations of Mycobacterium tuberculosis (Mtb) antigens (Ag) in direct, unaltered clinical specimens such as blood, urine and saliva.

This easy-to-use assay can be used to diagnose active tuberculosis (TB) disease in patients in POC settings in ˜45 minutes. This assay also is also useful for monitoring TB treatment, to quantify the reduction of Mtb Ag in clinical samples over the course of treatment. The assay has no moving parts, requires only minimal, low-cost fabrication and has an estimated cost-of-goods <3 USD/test.

The novel ECIA approach/assay presented in the present disclosure lays the foundation for a rapid, low-cost POC diagnosis of COVID-19, tuberculosis, and/or other infectious diseases directly from saliva, and will have a sustained positive impact on the COVID-19 pandemic as well as infectious diseases caused by emerging pathogens by enabling broad uptake of low-cost, rapid and repeated viral and bacterial antigen testing in a diversity of decentralized settings, which can significantly reduce transmission risks. The disclosed ECIA approach/assay could also transform the COVID-19 testing landscape from high-cost, centralized, population-level testing to low-cost, on-demand point of care (POC) testing, which can assist with public health measures in developing countries where vaccine distribution is scarce and/or uneven.

### Rapid Assay Technique

### Test Strips

In one aspect, disclosed herein is a test strip for detecting a disease or monitoring treatment of a disease in a subject, the test strip including a working electrode and a combination reference and counter electrode, wherein the working electrode and combination reference and counter electrode include a substrate coated with metal, and wherein the metal is bonded to a detection antibody for an antigen associated with the disease. Further in this aspect, the metal can be a thin film. In some aspects, the thin film can be applied by sputtering. In one aspect, the metal can be gold. In one aspect, the substrate can be glass or can be a plastic material such as, for example, polyethylene terephthalate glycol (PETG), polybutylene terephthalate (PBD), polyethylene terephthalate (PET), ethylene vinyl acetate (EVA), acrylonitrile butadiene styrene (ABS), polytetrafluoroethylene (PTFE), polyamide, polyetheretherketone (PEEK), polycarbonate, polyethylene terephthalate polyester (PETP), high density polyethylene (HDPE), low density polyethylene (LDPE), linear low density polyethylene (LLDPE), polymethylpentene (PMP), poly(p-phenylene oxide) (PPO), polypropylene, high impact polystyrene (HIPS), polyvinyl chloride (PVC), styrene acrylonitrile (SAN), acrylonitrile styrene acrylate, polyvinyl alcohol (PVOH), or any combination thereof.

In any of these aspects, the detection antibody can be coupled to the metal of both electrodes using carboxyl-amine coupling, a thiolate self-assembled monolayer (SAM), 4-carboxymethylaniline conjugation, or any combination thereof.

In one aspect, the disease can be tuberculosis or can be COVID-19, or another infectious disease caused by a bacterium, fungus, or virus. In one aspect, the disclosed test strips are not cross reactive with antigens from diseases not being detected or monitored such as, for example, influenza, respiratory syncytial virus, parainfluenza, rhinovirus/enterovirus, adenovirus, bordetella, or another human coronavirus. In one aspect, the subject is a mammal such as, for example, a human or a bovine.

In some aspects, when the disease is tuberculosis, the capture antibody can be selected from an anti-CFP-10 (i.e., 10 kDa culture filtrate protein) antibody, an anti-ESAT-6 (6 kDa early secreted antigenic target) antibody, an anti MPT-64 antibody, an anti-Ag85B antibody, an anti LAM antibody, or any combination thereof. In one aspect, using a combination of antibodies for different antigens can enhance specificity and/or accuracy of detection. In another aspect, when the diseases is COVID-19, the capture antibody can be an anti-receptor binding domain (anti-RBD) antibody, a 51 spike subunit antibody, an anti-nucleocapsid antibody, or any combination thereof. In any of these aspects, the capture antibody can have a capture efficiency of at least 50%.

In any of these aspects, the test strips are robust under standard storage conditions. In one aspect, the capture antibody retains at least 85%, at least 90%, or at least 95% of its initial activity level after storage. In one aspect, storage at a low temperature such as, for example, 4° C., may be helpful in retaining antibody activity. In another aspect, the test strips bonded to capture antibodies are stable for at least 2 weeks or for at least 30 days. In a further aspect, the test strips are inexpensive and are disposable after use.

### Methods for Detecting Diseases and Monitoring Treatment of Diseases

In another aspect, provided herein is a method for detecting a disease in a subject, the method including at least the following steps:
(a) incubating the test strip disclosed herein with a biological sample from the subject;
(b) rinsing the test strip to remove unbound material;
(c) incubating the test strip with a detection antibody specific to at least one disease-related antigen, the detection antibody including at least one tag;
(d) rinsing the test strip to remove unbound detection antibody; and
(e) detecting a signal from the at least one tag.

In another aspect, the disease can be tuberculosis, COVID-19, or another disease, and the biological sample can be blood, serum, saliva, or urine. In any of these aspects, the test strip is not cross reactive with matrix materials from the biological sample. In one aspect, the biological sample can be used directly to contact the test strip after collection of the biological sample and without further processing such as, for example, centrifugation, desalting, solid phase extraction, or the like. In one aspect, the biological sample can have a volume of from about 1 μL to about 100 μL, or of about 1, 5, 10, 15, 20, 25, 50, 75, or about 100 μL, or a combination of any of the foregoing values, or a range encompassing any of the foregoing values. In some aspects, the entire biological sample can be used to contact the test strip, and in other aspects, a smaller portion of the biological sample can be used to contact the test strip.

In any of these aspects, the biological sample can be incubated with the test strip for from about 10 minutes to about 1 hour, or for 10, 15, 20, 25, 30, 35, 40, 45, 50, or about 55 minutes, or about 1 hour or a combination of any of the foregoing values, or a range encompassing any of the foregoing values. In another aspect, the test strip can be incubated with the detection antibody for from about 30 minutes to about 1 hour, or for 30 or 45 minutes or 1 hour, or a combination of any of the foregoing values, or a range encompassing any of the foregoing values.

In another aspect, the at least one tag can be horseradish peroxidase (HRP), an enzyme label, or any combination thereof. In another aspect, when the at least one tag is HRP, the method further includes contacting the test strip with a solution of 3,3′,5,5′-tetramethylbenzidine (TMB) and hydrogen peroxide. In a still further aspect, a current can be applied to the test strip to initiate production of the amperometric signal from the HRP tag. In one aspect, the applied current can have a magnitude of about −0.1 V or another signal chosen to correspond to the particular tag being used in for a given test strip.

In one aspect, the signal from the at least one tag can be an amperometric signal. In another aspect, the signal can be detected using a potentiostat or potentiometer. In some aspects, the potentiostat or potentiometer can be mobile. In any of these aspects, the magnitude of the amperometric signal can correspond to a concentration of the antigen in the biological sample. In another aspect, the magnitude of the amperometric signal is directly proportional to the amount of HRP-tagged antibody bound to the test strip.

In an aspect, redox cycling can be performed to amplify the signal. In another aspect, the method can detect a concentration of at least 500 pM of antigen in the biological sample. In another aspect, when the diseases is a viral disease, the method can detect from about 5 to about 5×10⁸ viral particles in the biological sample, or about 5, 50, 500, 5000, 5×10⁴, 5×10⁵, 5×10⁶, 5×10⁷, or about 5×10⁸ viral particles in the biological sample, or a combination of any of the foregoing values, or a range encompassing any of the foregoing values. In any of these aspects, the method can be completed in under 45 minutes, or can be completed in under 90, 75, 60, 45, or 30 minutes, or a combination of any of the foregoing values, or a range encompassing any of the foregoing values, and/or can be completed on-site at a point of care (POC) for the subject.

In one aspect, the absence of the at least one signal indicates that the subject does not have the disease.

In another aspect, the method includes quantifying an amount of the at least one disease-related antigen in the biological sample.

Also disclosed is a method for monitoring treatment of an infectious disease in a subject, the method including at least the following steps:
(a) performing the disclosed method outlined above in a subject having the infectious disease a first time and obtaining a first antigen quantity;
(b) treating the infectious disease; and
(c) performing the disclosed method outlined above in the subject a second time and obtaining a second antigen quantity;
wherein successful treatment of the infectious disease is indicated by a lower second antigen quantity relative to the first antigen quantity.

### Tuberculosis Assay

Current diagnostic methods endorsed by the World Health Organization range from imaging (e.g., chest X-ray) and culture-based techniques to nucleic acid amplification tests and antigen detection techniques (e.g. via LAM, or lipoarabinomannan, lateral flow). Specificity of these tests is generally high, at 90% or above, but sensitivity can be lower and/or unpredictable (e.g. 32-94% for a conventional sputum microscopy smear or 44-54% for the LAM lateral flow assay). Furthermore, these tests can require expensive equipment with high operator training requirements to perform, and obtaining results can take days to weeks, which may not be helpful in monitoring disease and treatment progress. Existing tests often require sputum, which may be difficult for children to produce, or which may have a lower bacterial load in the case of patients living with HIV.

In one aspect, the present disclosure provides a disposable screen-printed dual carbon electrode sensing chip to capture both ESAT-6 and CFP-10 antigens from Mycobacterium tuberculosis in saliva and other clinical samples. A single dual sensor prototype is created, first by optimizing probes separately, one with ESAT-6 and one with CFP-10, then combining both probes on a single chip to create an assay that is able to simultaneously measure these TB specific antigens. ESAT-6 and CFP-10 antibodies are attached to the substrate to capture antigens from the sample. A final wash with the antibodies containing flags allows for detection of the presence of the specific antigen.

Electrochemical immuno test strips useful herein consist of thin-film sputtered gold electrodes on a PETG plastic substrate as working/counter/reference electrodes on which are carried out a sandwich format-based immunoassay using HRP attached to the detection antibody (DAb) as the enzyme label and H₂O₂/TMB as the enzymatic substrate/mediator system for electrochemical transduction. In some aspects, the electrodes are first sputtered with chromium or another metal and then sputtered with gold. To ensure the capture of the target antigen from the sample, the Au working electrode is modified with the specific anti-analyte antibody by covalent attachment through an optimized thiolate self-assembled monolayer formed on the Au surface. Such surface chemistry, together with the treatment with a blocking agent, can both suppress the non-specific adsorption effects and to achieve remarkably high sensitive antigen detection. Other electrode surface modification functionalization (e.g. 4-carboxymethylaniline) and enzyme labels compatible with these approaches can also be used for satisfactory electrochemical immunoassay performance.

To carry out the disclosed bioassays, a microliter droplet of the selected biofluid is placed on a immunostrip to cover the capture antibody modified electrode area. This is followed by immuno-reaction with the target antigen and the HRP-labeled Ab, to form a sandwich immunocomplex. Then, TMB/peroxide detection solution is introduced. HRP thus catalyzes the reduction of H₂O₂ coupled to the oxidation of TMB into its oxidized form. The corresponding H₂O₂ reduction signal at −0.1 V (vs. Ag/AgCl) is thus directly proportional to the amount of the captured HRP tag and hence to the concentration of the target antigen.

The results demonstrate that the CAb and the CFP-10 protein can be satisfactorily immobilized on an Au electrode and detected using a potentiostat.

### SARS-CoV-2 Assay

During spikes in viral transmission, COVID-19 testing capacity in the US has been hovering somewhat below what experts estimate is an “adequate” level of testing. A lack of both capacity and diversity of testing solutions is the primary reason for this deficiency; which is sustaining transmission and driving up daily deaths, especially as new SARS-CoV-2 variants emerge and spread. While several rapid, real-time PCR (RT PCR) platforms have recently approved for point-of-care (POC) COVID-19 diagnosis, the vast majority of the diagnostic burden is still carried by centralized RT PCR detection of SARS-CoV-2 (CV2). While RT PCR is a necessary component of the US and global testing strategy, the complexity of RT PCR solutions and instruments, as well as supply chain obstacles and high costs, make it highly unlikely this approach is sufficient to meet the massive and urgent demand for cheap, rapid, decentralized repeat testing in the US and the world. The approach and assay of the present disclosure can transform the COVID-19 testing landscape from high-cost, centralized, population-level testing to low-cost, on-demand POC testing, which may be especially helpful in developing countries where vaccines are not widely available.

The present disclosure thus provides a novel Electrochemical Immunoassay (ECIA) to detect CV2 Spike (S1) antigen directly from unprocessed patient saliva. In one aspect, the rationale for using ECIA for this novel assay, is that while lateral flow assays (LFA) have a huge cost and simplicity advantage over RT PCR, the only two COVID-19 LFAs to receive EUA to date (Quidel, BD) are severely limited by low sensitivity (<85%). The solution provided in the present disclosure has significant advantages over POC RT PCR and FDA approved LFAs. In one aspect, the present disclosure provides that 1) the final test, based on a disposable ECIA sensor, can be manufactured and produced at massive scale with cost-of-goods (COG) of about $2/sample; 2) the ECIA format enables receiving test results in about 15 minutes in Clinical Laboratory Improvement Amendments (CLIA) Certificate of Waiver settings; 3) 10-100 viral copy limit of detection (LoD) and sensitivity/specificity approaching RT-PCR can be achieved; 4) the ECIA assay can be quantitative, with potential for POC diagnosis and treatment monitoring in decentralized healthcare settings.

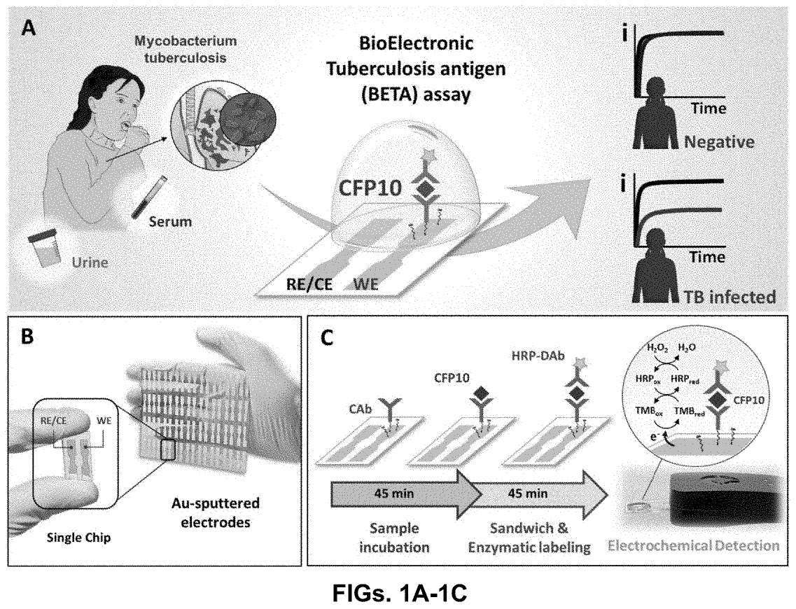

In certain embodiments, the ECIA approach (FIGS. 1A-1C) exploits an immunosensing technology that combines advanced surface chemistry with powerful signal amplification for detecting ultra-low concentration biomolecules, directly from 1-10 μL of saliva. In another aspect, preliminary data from contrived samples (saliva spiked with recombinant S1 protein from a Wuhan strain) indicate that the ECIA assay can reproducibly detect as little as 250 pM of antigen in this sample matrix. Thus, in a further aspect, the ECIA approach can enable detection of CV2 in unprocessed clinical saliva samples with a limit of detection (LoD) of 10-100 viral copies per test reaction, and achieve a sensitivity exceeding 90% and a specificity approaching 100% compared to RT PCR reference results.

In one aspect, to evaluate a) the analytical specificity of the prototype assay; b) the potential for saturation (high-dose Hook effect); c) potential interference and inhibitory effects of the sample matrix; and d) the stability of the sensor and antibodies, the present disclosure provides that the CV2-specific capture and detection antibodies, with high affinity for the unique receptor binding domain of the CV2 S1 protein, do not have sufficient homology or affinity with other pulmonary pathogens to cause false positive results. In a further aspect, however, the technology can be modified to incorporate antibodies for other pathogens, allowing the rapid development of tests for emerging and future diseases.

To determine the LoD of the novel ECIA assay in saliva using quantified, inactivated whole CV2 virus spiked into disease-free saliva, the present disclosure provides that, using FDA recommended methods (2 fold dilution series×3 replicates each), the ECIA assay enables to reproducibly detect as few as 10-100 viral copies in a 10 μL saliva sample.

In one aspect, a minimum of 90% positive percent agreement and 98% negative percent agreement with clinical-grade RT PCR test results were achieved. Thus, the ECIA approach and/or assay of the present disclosure lay the foundation for rapid, low-cost POC diagnosis of COVID-19 directly from saliva. In a further aspect, the present disclosure will have a sustained positive impact on the COVID-19 pandemic, especially in countries lacking healthcare infrastructure and adequate vaccine distribution, by enabling broad uptake of low-cost, rapid and repeated COVID-19 testing in a diversity of decentralized settings, which can significantly reduce transmission risks.

In another aspect, the diagnostic solution disclosed herein also has the potential to quantify CV2 antigen, which may be useful for estimating viral load and measuring viral decline in order to monitor and manage transmission risk. In one aspect, currently only RT PCR can currently provide accurate estimates of viral load, but estimates based on quantifying extracted CV2 RNA are complicated by non-viable RNA. The disclosed ECIA assay, designed to estimate viral load from CV2 spike protein quantification, can be better associated with viable viral load.

In a further aspect, the single-step incubation process (sample+detection antibody applied directly to sensor) dramatically reduces workflow complexity, reagent requirements and time to result.

In still another aspect, the disposable ECIA sensor consists of two gold sputtered electrodes on plastic substrate, fabricated using a photolithography-free masking method, thus enabling a cost of approximately $2/test at scale.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an antibody,” “a respiratory disease,” or “a biological sample,” includes, but is not limited to, mixtures or combinations of two or more such antibodies, respiratory diseases, or biological samples, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “diagnose” and “diagnosis” refer to detection and/or identification of a disease in a subject. In one aspect, diagnosis proceeds by using the methods disclosed herein to determine whether or not a subject suspected of having a specific disease, actually has that disease. If the test strips disclosed herein generate no signal using the disclosed methods, the subject is not diagnosed with the specific disease and the subject should be assessed for the presence of other diseases causing the same or similar symptoms.

As used herein, the terms “treating” and “treatment” can refer generally to obtaining a desired pharmacological and/or physiological effect. The effect can be therapeutic in terms of a partial or complete cure of a disease, condition, symptom or adverse effect attributed to the disease, disorder, or condition. The term “treatment” as used herein can include any treatment of tuberculosis, COVID-19, and/or another infectious disease in a subject, particularly a human, and can include any one or more of the following: (a) inhibiting the disease, i.e., arresting its development and (b) relieving the disease, i.e., mitigating or ameliorating the disease and/or its symptoms or conditions. As used herein, the term “treating”, can include inhibiting the disease, disorder or condition, e.g., impeding its progress; and relieving the disease, disorder, or condition, e.g., causing regression of the disease, disorder and/or condition, or can include eliminating the infectious organism causing the disease from the subject.

As used herein, “infectious disease” refers to a disease caused by pathogenic agents or microorganisms in the body. In one aspect, the pathogenic agents or microorganisms can be bacteria, fungi, viruses, or a combination thereof.

As used herein, “RT-PCR” refers to “real-time polymerase chain reaction,” a technique that can be used quantitatively or semi-quantitatively to monitor the amplification of a target DNA molecule during PCR. RT-PCR can be used with non-specific fluorescent dyes or sequence-specific DNA probes labeled with a fluorescent reporter. In some aspects, COVID-19 testing can be performed by RT-PCR. However, results from this type of testing can have a slow turnaround time, which may lead to further spread of infectious diseases as patients may not adequately quarantine or isolate while waiting for results. Additionally, RT-PCR tests can require expensive reagents and analytical equipment to complete. In one aspect, the disclosed methods and test strips are faster and less expensive than RT-PCR while providing comparable levels of accuracy.

The test strips useful herein include a “capture antibody” (CAb) bound to the surface of the test strips. In one aspect, the capture antibody is specific to an antigen to the disease being tested for with the disclosed test strips (for example, ESAT-6 or CFP-10 for tuberculosis, or spike protein for COVID-19). When the antigen contacts the capture antibody, it is immobilized on the test strip.

As used herein, “antigen” refers to a molecule or portion of a molecule from an infectious organism or virus that can trigger an immune response in an individual. In one aspect, antibodies in the disclosed test strips can bind to selected antigens; this binding can be used to detect the presence of or diagnose an infectious disease in a subject.

The methods disclosed herein also make use of a “detection antibody” (DAb). In one aspect, once the antigen is bound to the capture antibody which is immobilized on the disclosed test strips, a solution including the detection antibody can be applied to the test strips. The detection antibodies useful herein bind to the immobilized antigens. In some aspects, the detection antibody can include a tag such as, for example, HRP, that can be used to generate a signal, wherein the signal is proportional to the amount of detection antibody, and hence the amount of antigen, in a biological sample from a subject.

“Horseradish peroxidase” or HRP as used herein is a metalloenzyme that catalyzes the oxidation of organic substrates by hydrogen peroxide. In one aspect, HRP can be used as a tag on the detection antibodies useful herein. Further in this aspect, when the detection antibodies bind to the antigens immobilized by the capture antibodies on the substrate, an organic molecule such as, for example, TMB and hydrogen peroxide can be placed on the disclosed test strips. Oxidation of TMB can be detected as an amperometric signal when a current is applied to the test strip; this amperometric signal is proportional to the amount of antigen in the biological sample.

As used herein, an “amperometric signal” or “amperometric response” refers to a current obtained due to a reduction or oxidation reaction when a potential is applied to a disclosed test strip. Amperometric signals are directly proportional to the amount of analyte (e.g. antigen) in a biological sample applied to the test strip and can be used to diagnose an infectious disease in a subject. In one aspect, absence of an amperometric signal in the disclosed methods indicate that the subject does not have the disease.

As used herein, “redox cycling” refers to self-regenerating the substrate of an enzyme tag in order to amplify an analytical signal. In one aspect, 2 neighboring electrodes can be used for redox cycling. In an alternative aspect, an additional enzyme can be used for enzymatic recycling.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Sensor Fabrication

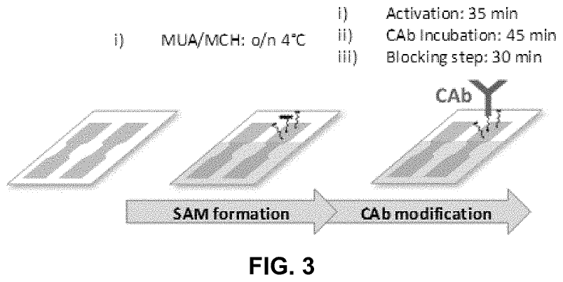

BETA assay sensors were manufactured in batches using a previously developed proprietary photolithography-free masking method, with each sensor consisting of two rectangular 21 mm2 area electrodes on a plastic substrate to create the working electrode (WE) and a joint reference/counter electrode (RE/CE). WEs for TB sensing were modified by immobilizing commercial-grade, Mtb-specific, anti-CFP-10 capture antibodies (CAb) onto the WE surface and “blocking” the surface to prevent non-specific binding (FIG. 3).

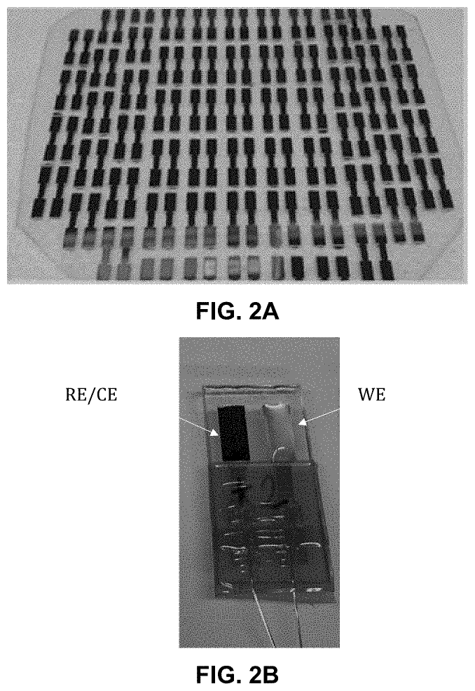

Assay sensors were manufactured in batches using a proprietary photolithography-free masking method. First, a CRICUT® (Cricut, Inc., South Jordan, Utah) machine was used generate the rectangular electrode patterns on the laminated protective cover of a polyethylene terephthalate glycol (PETG) plastic sheet (Small Parts Inc.). Then a “Denton Discovery 18 Sputter System” (Denton Vacuum, NJ), under direct current and with Argon gas, was used to sputter deposit Cr and then Au onto the PETG substrate. Each completed sensor consisted of two rectangular 21 mm2 area electrodes: a working electrode (WE) and a joint reference/counter electrode (RE/CE).1 The electrodes were immersed overnight at 4° C. in a 0.1 mM MUA/1.0 mM MCH (MUA=11-mercaptoundecanoic acid; MCH=6-mercapto-1-hexanol) solution to form a stable thiol self-assembly monolayer (SAM). The electrodes were then washed with pure ethanol and water. The resulting SAM-functionalized WEs were incubated with 10 μL of a 0.4 M EDC/0.1 M NHS solution (EDC=1-ethyl-3-(3-dimethylaminopropyl) carbodiimide hydrochloride; NHS=N-hydroxysuccinimide) to activate the terminal carboxylic groups. After washing three times with phosphate buffer (PB), the electrodes were dried with a compressed air gun. After sensor fabrication, the WEs were modified by covalently attaching anti-CFP10 antibodies or capture antibodies (CAb) on the electrode surface by incubating the electrodes with 10 μL of 100 μg/mL CFP10 CAb solution for 45 minutes. The electrodes were again washed three times with PB and dried with a compressed air gun. Remaining unreacted carboxylic groups were deactivated by incubating the WE with 10 μL of ethanolamine solution for 30 min. After a final PB wash and dry, the CAb-modified WE sensors were stored at 4° C. until use. Exemplary sensors are shown in FIGS. 2A-2B.

After sensor fabrication, the WEs are modified by immobilizing anti-CFP-10 capture antibodies (CAb) on the electrode surface. First, the electrodes are immersed overnight at 4° C. under humid conditions in a 0.1 mM MUA/1.0 mM MCH mixture solution (prepared in pure ethanol) to form a stable thiol self-assembly monolayer (SAM). The electrodes are then washed with pure ethanol and water sequentially for 10 minutes each.

The resulting SAM-functionalized WEs are incubated with 1 OA of a 0.4 M EDC/0.1 M NHS mixture solution (prepared in MES 25 mM, pH 6.5) for 35 minutes at room temperature under humid conditions to activate the terminal carboxylic groups. After washing three times with phosphate buffer (PB, 0.1 M, pH 7.4) the electrodes are gently dried with a compressed air gun. The capture anti-CFP-10 antibodies are then covalently attached by incubating the electrodes with 10 μL of 100 μg mL-1 CFP-10 CAb solution (prepared in MES 25 mM, pH 6.5) for 45 minutes. The electrodes are again washed three times with PB and gently dried with a compressed air gun. Remaining unreacted carboxylic groups are deactivated by incubating the WE with 10 μL of 2M ethanolamine solution (pH 8.5) for 30 min. After a final PB wash and gentle dry, the CAb-modified WE sensors are stored at 4° C. until use. Stability studies suggest current sensor is at a minimum, stable for 30 days at 4° C. A schematic of this process is shown in FIG. 3.

### Sensor Optimization and Testing

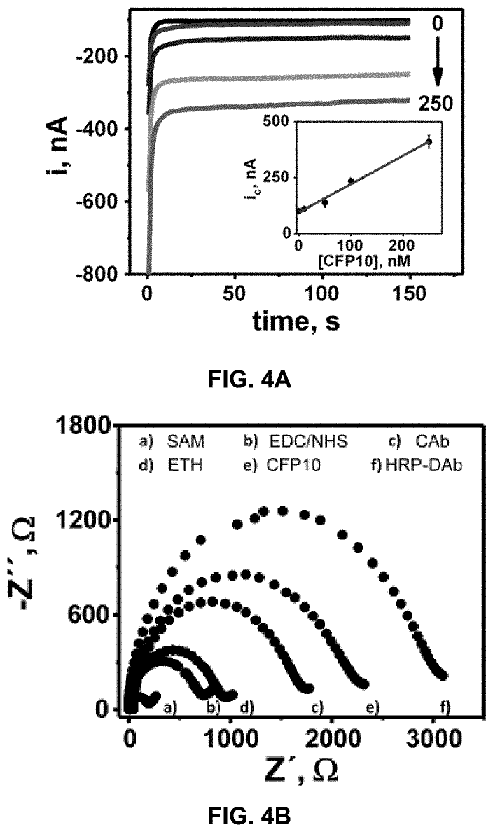

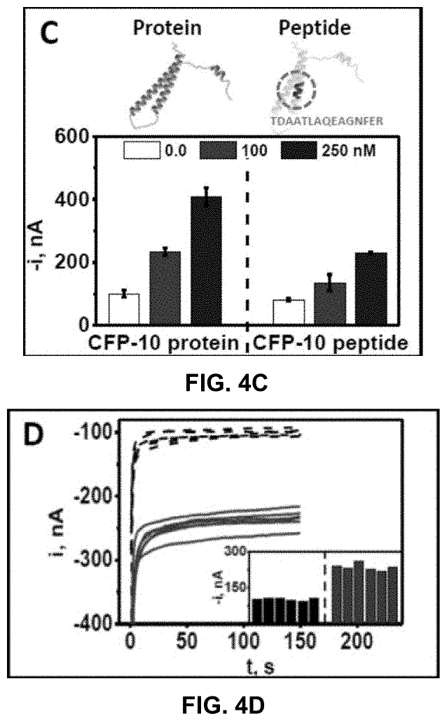

High sensitivity detection capability was achieved by comprehensive study and optimization of all the experimental variables affecting the BET antibody-modified sensor preparation and the bioassay protocol. These included composition of the binary self-assembled monolayer (SAM); concentrations of the 11-mercaptoundecanoic acid (MUA), 6-mercapto-1-hexanol (MCH) and ethanolamine reagents used to prevent non-specific adsorption; CAb and HRP-tagged Mtb CFP-10 detector antibody (HRP-DAb) concentration; number of assay steps; solution composition for enzymatic labeling and incubation; and incubation times for CAb, CFP-10 antigen and HRP-DAb binding. In order to identify the optimal approach, all of the sequential surface modification steps performed, from initial functionalization of the gold electrode to the final HRP-tag capturing on the sensor were monitored by Electrochemical Impedance Spectroscopy (EIS) using a Gamry “Interface 1010E” instrument (Gamry, PA) (See FIG. 4B).

### Example 2: Exemplary Workflow

An exemplary assay workflow using the disclosed sensors proceeds as follows:
1. Several drops of the clinical sample (e.g. urine or serum) are removed from the sample container.
2. One drop (about 10 μL) of the clinical sample is deposited on the WE and incubated for about 45 minutes. In one aspect, when the sensor is a TB-sensing device, this can serve to bind CFP-10 antigen to the CAb.
3. The WE is washed 3× with PB and dried using an air gun. In some aspects, this removes sample matrix and stops the immunoreaction of antigen with the CAb.
4. A 10 μL drop of HRP-DAb solution is placed on the WE and incubated for 45 min.
5. The WE is washed 3× with 0.05% sodium dodecyl sulfate (SDS) solution and 3× with PB to remove unbound HRP-DAb, followed by drying using an air gun.
6. The sensor is placed in a potentiometer. In some experiments, an EmStat3 Blue potentiometer can be used in conjunction with PSTrace software (Palmsens, The Netherlands).
7. A 20 μL drop of TMB/H₂O₂ is deposited across the WE and RE/CE. A current (−0.1 V) is applied and the results are recorded for 2.5 min.

In some experiments, the current measured from the potentiometer corresponds to the reduction of oxidized TMB generated by the coupled reaction of H₂O₂ and catalyzed by the HRP tag on the DAb, which is directly proportional to the amount of HRP-tagged DAb bound to the sensor and corresponds to the concentration of antigen (in this example, CFP-10) in the sample.

Incubation times, wash solutions, buffers, and the like, can change depending on the assay being developed of performed. A typical amount of operator time is about 1.5 minutes, total, with 90 minutes for incubation and 2.5 minutes for reading the sample, for a total of about 94 minutes from sample collection to availability of results.

### Example 3: Clinical Sample Acquisition

Clinical samples used in this study were obtained from three independent TB and drug resistant TB study cohorts from previously completed and ongoing TB studies. Samples were collected under each study's respective University of California San Diego IRB approved protocol, and bio-banked for future use. Informed consent was obtained at the time of specimen collection from all study participants. All study procedures were performed in accordance with approved protocols and relevant guidelines. In brief, participants in Cohort A were enrolled in an antigen diagnostic study in San Diego. Serum and urine samples were collected from patients presenting for acute care who were assessed for TB disease and underwent standard reference testing with culture and/or GeneXpert. Due to enrollment delays, some TB positive participants in this cohort initiated treatment prior to sample collection. Samples from Cohort B were collected from patients recruited in Moldova as part of a study designed to identify blood-based biomarkers of progression from not infected to active TB infection. Serum samples were collected from culture positive, index TB cases at treatment initiation, with some samples being collected post-treatment initiation. Samples included in Cohort C were collected from Moldovan participants enrolled in a study designed to evaluate a novel, molecular diagnostic for XDR-TB diagnosis. Study participants consisted of individuals who were considered at risk for drug resistance. Initial serum samples were collected at enrollment, before treatment, and then again at set intervals during treatment. Bio-banked serum samples from all cohorts and urine from cohort A were stored at −20° C. for weeks to years (3 years maximum) prior to subjecting them to BETA assay evaluation.

### Example 4: Optimization of Tuberculosis Antigen Testing

As the assay performance of bioelectronic immunoassays can be highly dependent on the biochemical makeup of the sample matrix, we established calibration curves first in phosphate buffer (PB) as sample matrix, then in serum and urine independently. For calibration of the assay in PB, concentration standards of recombinant Mtb CFP-10 antigen (ImmunoDx, MA) were spiked into PB (0, 10, 50, 100 and 250 nM). For calibration of the assay for use with serum and urine sample matrices, Mtb CFP-10 antigen was spiked in increasing concentrations (0, 1, 5, 10, 25 nM) into commercially acquired negative control, charcoal stripped (4×) human serum (Biochemed, VA) and pooled human urine (MyBioSource, CA).

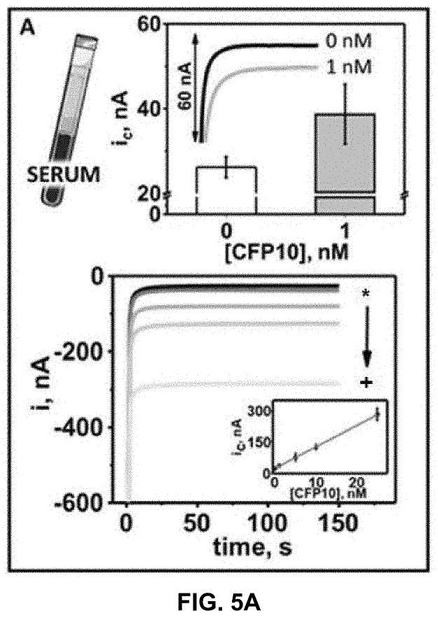

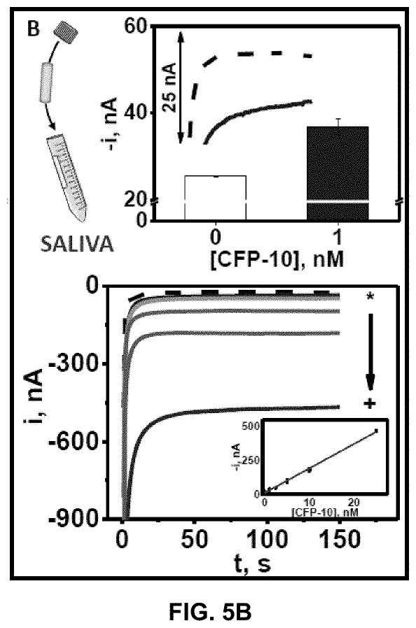

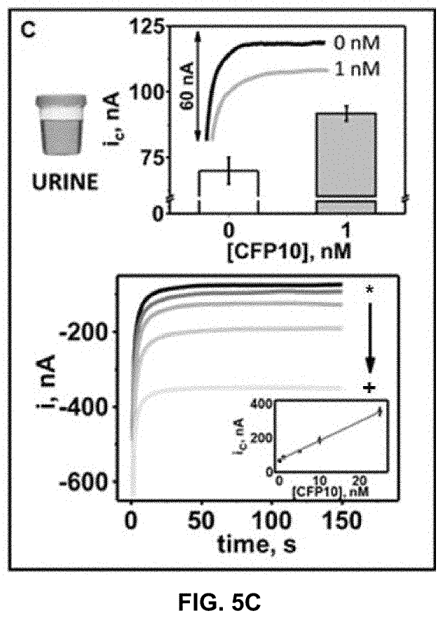

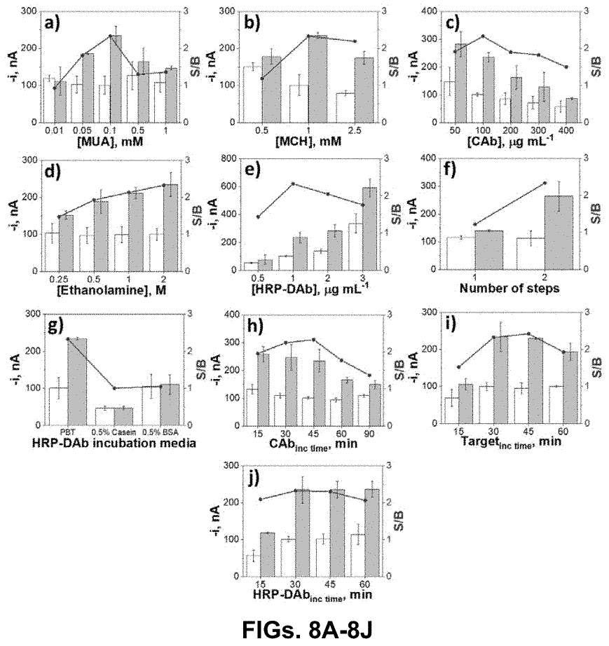

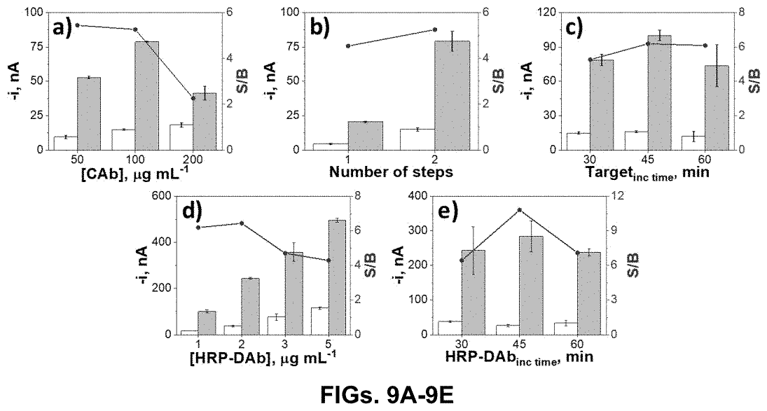

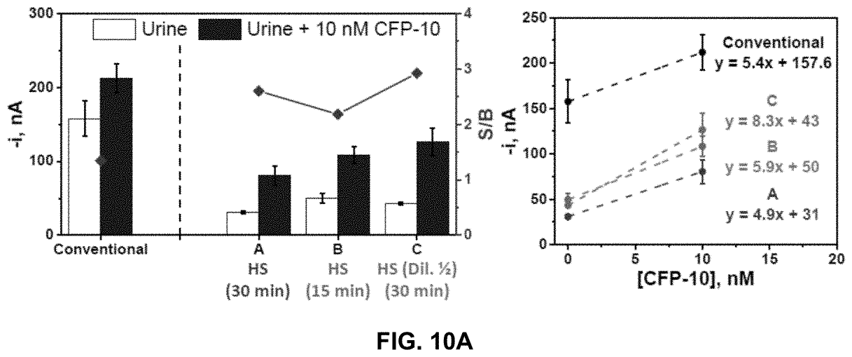

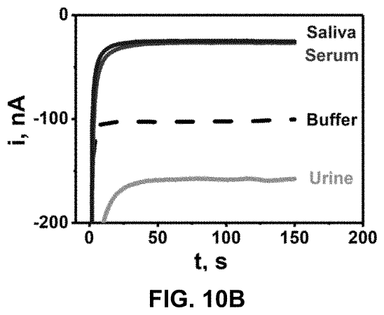

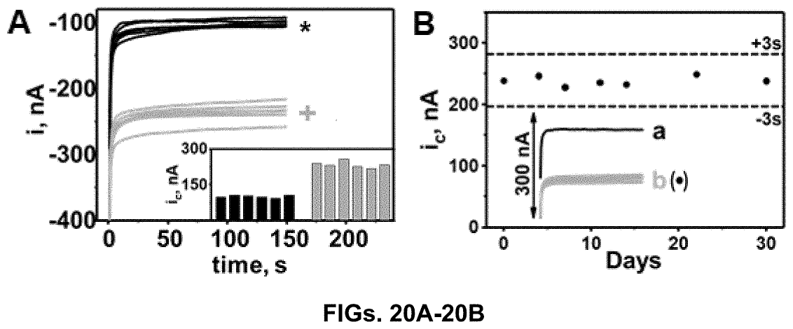

Original assay conditions were as follows: SAM formation (conventional); activation step: EDC/NHS (0.4 M/0.1 M), 35 min, CAb (200 μg/mL), 1 h; blocking step (see also FIGS. 10A-10B): ethanolamine (2 M), 30 min CFP-10 (10 nM), 1 h, DAb-HRP (2 μg/mL), 1 h; incubation of DAb-HRP in phosphate buffer containing 0.02% polysorbate 20; and incubation of DAb-HRP in PBS containing 1.0% casein. Optimization of variables in initial (FIGS. 4A-4D and 8A-8J) and subsequent (FIGS. 5A-5C and 9A-9E) tests are summarized in FIGS. 4A-4D and Table 1. See also FIGS. 20A-20B for data from optimization in buffer.

TABLE 1
Optimization of Variables

| | | | Re-Optimized in | |
|---|---|---|---|---|
| | Optimized in Buffer | | Serum^{a} | |
| | Evaluated | Selected | Evaluated | Selected |
| Variable | Range | Value | Range | Value |
| [MUA], mM | 0.1-1 | 0.1 | | |
| [MCH], mM | 0.1-2.5 | 1.0 | | |
| [CAb], μg/mL | 50-400 | 100 | 50-200 | 100 |
| CAb incubation time, min | 15-90 | 45 | | |
| Number of Steps | 1-2 | 2 | 1-2 | 2 |
| Target_{incubation timer} min | 15-60 | 30 | 30-60 | 45 |
| [Ethanolamine], M | 0.25-2 | 2 | | |
| Ethanolamine incubation | | 30 | | |
| time, min | | | | |
| [HRP-DAb], μg/mL | 0.5-3 | 1 | 1-5 | 2 |
| HRP-DAb incubation | 15-60 | 30 | 30-60 | 45 |
| time, min | | | | |
| HRP-DAb Incubation | PBD, 0.5% | PBT | | |
| Media | Casein, | | | |
| | 0.5% BSA | | | |

^{a}Serum is human serum stripped with charcoal 4×.

Analytical calibration curve characteristics of the optimized Bioelectronic TB (BET) assay for the detection of different concentrations of CFP-10 standards prepared in buffer, charcoal stripped (4×) human serum and pooled human urine are summarized in Table 2:

TABLE 2
Analysis of Data

| Variable | Buffer | Serum^{a} | Saliva | Urine |
|---|---|---|---|---|
| R² | 0.9914 | 0.9997 | 0.9968 | 0.985 |
| Slope, nA/nM | 1.3 ± 0.2 | 10.2 ± 0.2 | 18 ± 1 | 11.4 ± 0.3 |
| Intercept, nA | 95 ± 26 | 27 ± 2 | 13 ± 16 | 74 ± 3 |
| LOD, nM | 3 | 0.4 | 1.0 | 0.7 |
| LOD (pg)^{b} | 330 | 44 | | 77 |

^{a}Serum is human serum stripped with charcoal 4×.
^{b}Amount of CFP-10 antigen detected in a 10 μL simple droplet used for all assay runs.

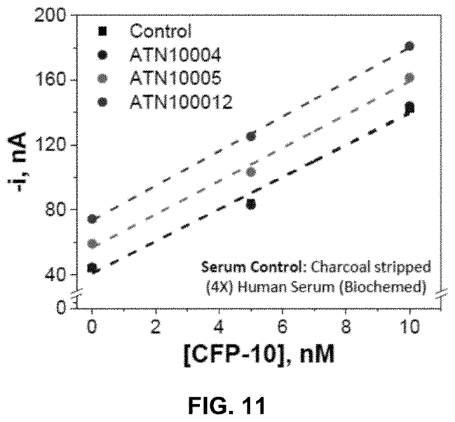

Matrix effects in several samples are summarized in FIG. 11 and Table 3:

TABLE 3
Matrix Effects in Human Biological Fluids

| Serum | Slope, nA/nM | tₑₓₚ |
|---|---|---|
| Control | 10.1 ± 0.1 | T_{tab(0.05; 4)} = 2.132 |
| ATN10004 | 9.9 ± 1.3 | 0.009 |
| ATN10005 | 10.2 ± 0.8 | 0.008 |
| ATN100012 | 10.6 ± 0.3 | 0.005 |

CFP-10 levels in selected samples are summarized in Table 4:

TABLE 4
CFP-10 Levels

| | CFP-10 Endogenous Levels, nM (n = 3) | | |
|---|---|---|---|
| Patient | Serum | Saliva | Urine |

| | |
|---|---|
| ATN10004 | Non-detectable |
| ATN10005 | 1.8 ± 0.3 |
| ATN100012 | 3.0 ± 0.9 |

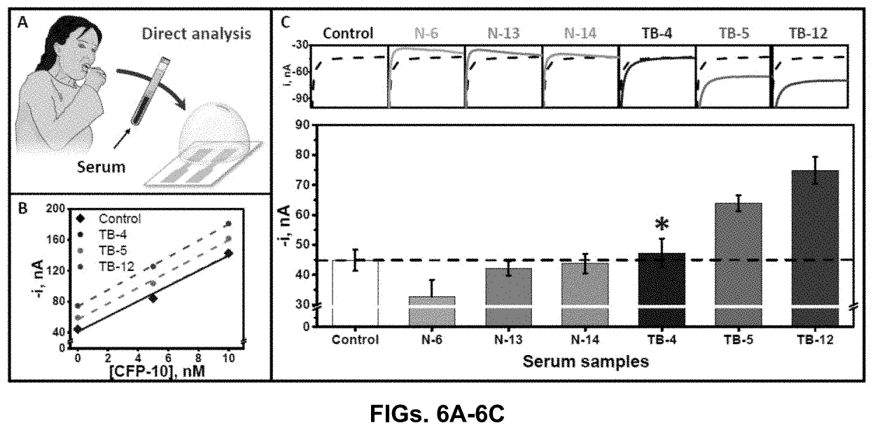

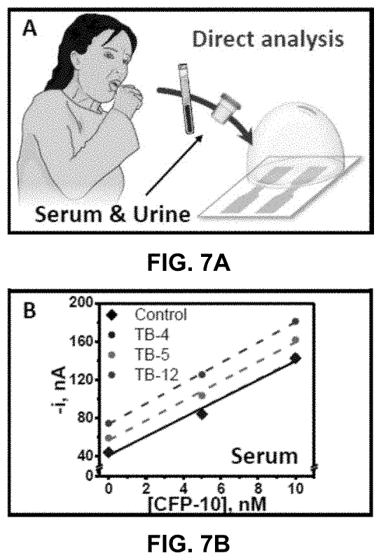

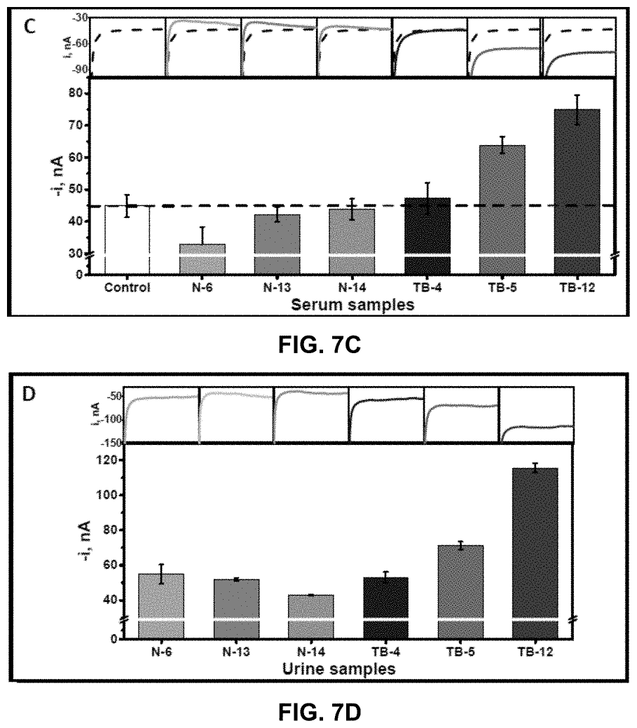

These results indicate the disclosed methods are reliable in serum samples and can be further optimized for different biological matrices. Testing in tuberculosis patients using various biofluid samples is shown in FIGS. 6A-6C and 7A-7D.

### Sensor and Assay Protocol Optimization

Sensor and assay parameters were assessed by comparing the ratio of amperometric results from positive control (“S”—signal) and negative control (“B”—blank) runs, optimizing for highest S/B ratio. FIGS. 8A-8J and 4B show the S/B ratios for each condition and final resulting Nyquist curves, respectively. Assay parameters were then re-optimized for serum matrix (FIGS. 9A-9E) with Table 1 showing final parameters selected for buffer and serum assays. Background amperometric responses of the serum-optimized assay for negative control buffer, serum and urine indicated that negative control serum had a nearly four-fold lower electrochemical background noise compared to buffer (FIGS. 10A-10B), likely due to partial electrode surface biofouling, typically observed in complex fluids. In contrast, negative control urine matrix produced higher electrochemical background signals, likely due to the higher non-specific adsorption of biomolecules present in urine (FIGS. 10A-10B). To reduce non-specific adsorption for the urine matrix, sensor electrodes were incubated for 30 minutes in Gibco® horse serum (ThermoFisher Scientific, MA) as a pre-conditioning step before running urine samples. This significantly improved the S/B ratio for urine (see FIGS. 10A-10B).

### Sensor Reproducibility and Stability

Reproducibility of the optimized BETA assay was evaluated by comparing chronoamperometric measurements from 12 optimized BETA sensors, batch-fabricated and run using buffer (n=6 replicates), and buffer spiked with 100 nM CFP-10 antigen (n=6). Storage stability of the sensors with bound CAb was assessed by batch-fabricating sensors and binding the CAb, then storing them at 4° C. and testing sensors on seven separate days over 30 days. Assays were run in triplicate with 100 nM Mtb CFP-10 spiked into PB and amperometric readouts were averaged and recorded.

The relative standard deviation (RSD) of amperometric results from a batch of 12 optimized sensors run with positive control (6×100 nM of CFP-10 in PB) and negative control samples (6×PB), was 5.9% and 5.2% respectively (FIG. 20A) indicating good reproducibility of the assay. Sensor stability was assessed periodically over a 30 day period. Amperometric responses ranged from 228.4 nA to 246.9 nA, and no systematic decrease was observed, indicating acceptable stability during observation period (see FIG. 20B).

### Assay Calibration and Limit of Detection

Calibration curves for the optimized assay in buffer (FIG. 4A), serum and urine (FIGS. 5A and 5C) to establish matrix-specific baseline analytical performance. See Table 2 for resulting calibration curve parameters. Based on these calibration curves, the estimated LOD for detection of Mtb CFP-10 antigen in serum was 0.4 nM (44 pg in a 10 μL clinical sample droplet) and 0.7 nM in urine (Table 2).

### Final Assay Protocol

The final prototype assay, optimized for serum and represented in a simplified schematic (FIGS. 1A-1B) is as follows: 1) 10 μL of serum was deposited on the modified WE and incubated for 45 minutes to bind CFP-10 antigen to the CAb; 2) A PB washing step (repeated 3 times) was used to remove sample matrix and stop the immunoreaction; 3) 10 μL of commercial-grade, horseradish peroxidase (HRP) labeled anti-CFP-10 detector antibody (HRP-DAb) solution was then placed on the WE and incubated for 45 minutes to sandwich the CFP-10 between the CAb and DAb on the WE; 4) A 0.05% SDS washing step (repeated 3 times), and another PB washing step (repeated 3 times) was used to remove unbound HRP-DAb; 5) The BETA sensor was then inserted into an “EmStat3 Blue” potentiometer, attached to a laptop running “PSTrace” software (Palmsens, The Netherlands); 6) A 204 drop of 3,3′,5,5′-tetramethylbenzidine (TMB)/H₂O₂ was placed over both electrodes, and a current of −0.1 V was applied and the amperometric signal was recorded for 2.5 minutes. The current measured from the potentiometer corresponded to the reduction of the oxidized TMB, generated by the coupled reduction of H₂O₂ catalyzed by the HRP tag on the DAb, which was directly proportional to the amount of the HRP-tagged DAb bound to the sensor, corresponding to the concentration of CFP-10 in the sample.

### Statistical Analysis

Clinical samples were evaluated in triplicate; error bars were estimated as standard deviation of the three replicates. Limit of Detection (LOD) was estimated using the 3 Sb/m criterion, where Sb is the standard deviation for 10 blank signal measurements and m is the slope value of the calibration plot. EIS data from sensor and protocol optimization was processed into Nyquist curves using “Gamry Echem Analyst” (Gamry, PA). Linear regression analyses were performed and concentration curves generated using “STATA 15” (College Station, Tex.) and “Origin” (OriginLab, MA).

### Example 5: Specificity of Sensors for SARS-CoV-2

The CV2-specific capture and detection antibodies, with high affinity for the unique receptor binding domain of the CV2 spike protein, do not have sufficient affinity for other pulmonary pathogens to cause false positive results.

### ECIA Sensor and Assay Workflow

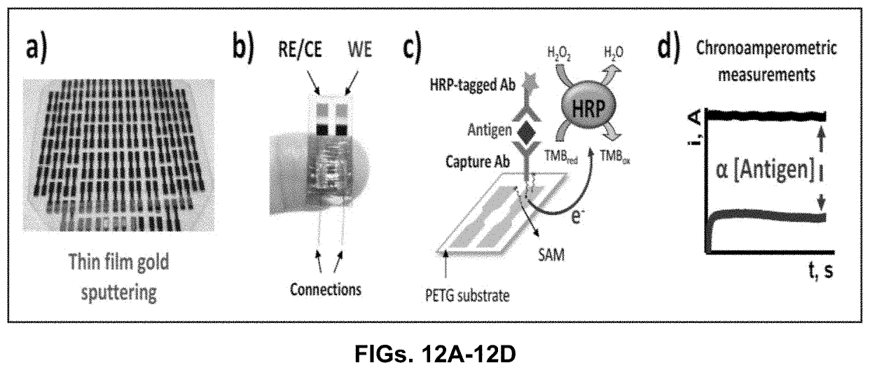

The prototype ECIA sensor consists of two gold (Au) sputtered electrodes on a polyethylene terephthalate glycol (PETG) plastic substrate fabricated using the proprietary photolithography-free masking method. One electrode acts as working electrode (WE) where antigen capture and detection occur, while the other electrode serves as a joint reference and counter electrode (RE/CE). Sensors are manufactured by covalently attaching capture antibodies to the self-assembled monolayer (SAM)-functionalized gold surface through carboxyl-amine coupling. A clinical sample volume of 10 μL is mixed with antigen-specific, horseradish peroxidase (HRP)-tagged detection antibodies, then placed on the WE probe and incubated for 15-20 minutes, allowing the target antigens to become sandwiched between the capture and detection antibodies. After 3,3′,5,5′-tetramethylbenzidine (TMB)/hydrogen peroxide (H₂O₂) solution is placed on the sensor, the HRP acts as a catalyst for the reduction of H₂O₂ and oxidation of TMB. This reaction produces a cathodic current signal (vs. colorimetric of LFAs) that is directly proportional to the amount of the captured HRP tag on the sensor and corresponds to the concentration of target antigen in the sample (FIGS. 12A-12D). Ultra-high sensitivity is achieved by optimization of the surface chemistry and minimizing non-specific adsorption effects. Redox cycling can also be utilized for further signal amplification as needed.

### Saliva as a Clinical Sample for Antigen Detection

Based on a comprehensive review of the literature, it appears that while there is a wide range of viral loads in clinical saliva samples from patients with COVID-19, it is not always lower than nasopharyngeal swab, as initially thought, and ranges from ˜10⁴ to 10¹⁰ virus copies/mL. Given that reaction volumes on the ECIA sensor from 10-100 μL of sample were accommodated—absolute viral loads in saliva samples were expected to range from 100 to 10⁸ copies/reaction. The sensor surface antibody density is far in excess of this (˜4.76 μg/cm2) and assuming capture efficiency of 50% (based on the published antibody K_{D}), between 5 and 5×10⁶ viral particles could be detected with the sensor. The HRP-based reaction also creates multiple signaling molecules (TMB⁺) for each viral particle, which further amplifies the signal. While this alone is sufficient to achieve optimal sensitivity, redox cycling can also be performed by which each signaling molecule is reversibly oxidized and reduced at corresponding neighboring electrodes, if needed. This will yield another 10-100× higher sensitivity.

### Antigen Target and Antibody Selection

Antigen/antibody selection is critical to both the sensitivity and specificity of the prototype CV2 ECIA assay. Ideal antigen targets include epitopes that are: i) accessible on the exterior of the virus; ii) plentiful; iii) unlikely to change/evolve over time; and iv) highly specific to the target virus. A comprehensive review was conducted of the most promising CV2 antigen targets and commercially available monoclonal antibodies that would yield best performance results with the ECIA and be most pragmatic from a manufacturing and supply chain perspective. It was also determined that the receptor binding domain (RBD) or entire S1 subunit of the spike (S) protein was be the best CV2-specific antigen target for this ECIA assay. The RBD of the S1 subunit has very high affinity for the human ACE2 receptor and is critical for binding and entry into host cells. This domain is also highly conserved due to its functional significance and is likely to remain stable as the virus evolves over time. Additionally, epitopes in the RBD can be bound to antibody without manipulating the virus as they have some of the highest surface accessibility scores relative to other S1 domains. This is in contrast to the nucleoprotein that other CV2 antigen tests (e.g. Quidel) detect, which is in the interior of the virus particle and requires an extraction step to release the antigen.

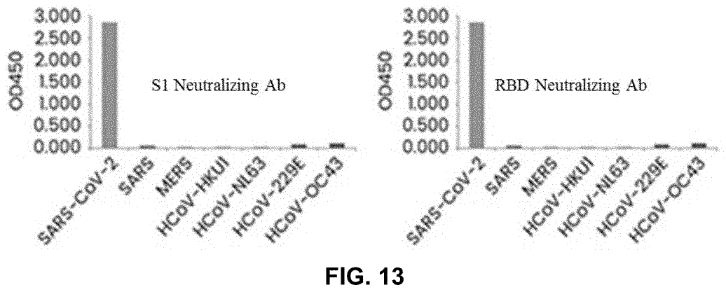

The RBD domain of S1 has approximately 74% amino acid sequence homology with SARS-CoV-1 (CV1), but only 20% homology with another human coronavirus (HCoV-NL63), and no homology with other human coronaviruses as they do not bind host cells via the ACE2 receptor. This indicates a low likelihood of weak cross-reactivity of CV2 anti-S1(RBD) Ab to CV1, but very low to zero likelihood of even weak cross-reactivity with other human coronaviruses or other potential pathogens that might be present in human saliva. This was confirmed by affinity studies conducted by Sinobiological (US) on anti-S1 and anti-S1(RBD) antibodies used in the assay (FIG. 13). Based on these preliminary studies, the prototype assay using commercially sourced monoclonal anti-S1(RBD) capture antibody (Sinobiological, PA) bound to the ECIA sensor and another commercially sourced monoclonal, HRP-tagged anti-S1 for the detection antibody (Cusabio, TX), was designed.

### Cross Reactivity Testing

The FDA EUA template for CV2 antigen tests recommends that CV2 antigen assays be tested for cross-reactivity against a broad range of pathogens that could be present in the clinical sample matrix including strains of: influenza, respiratory syncytial virus, parainfluenza, rhinovirus/enterovirus, adenovirus, bordetella, human coronavirus, and Mycobacterium tuberculosis; while the preliminary TTP criteria from WHO emphasize only the need to demonstrate no cross-reactivity with the most prevalent human coronaviruses. Based on the low likelihood of either of the anti-S1(RBD) and anti-S1 antibodies having affinity for any of the large diversity of pathogens listed by FDA, the analytical specificity testing was focused on the pathogens that are most likely to be present in high viral load in patients presenting for CV2 testing (influenza) and the only human corona virus known to have minimal homology with the CV2 S1 RBD (HCoV-NL63).

In these studies, two contrived samples experiments are conducted: 1) disease-free saliva spiked with inactivated Influenza A and B virus (Zeptometrix); 2) disease-free saliva spiked with inactivated HCoV-NL63 (ATCC). Each experiment is run using the workflow described above in “ECIA Sensor and Assay Workflow”. Experiments are run in triplicate and amperometric readings are collected using a hand-held EmStat3 Blue wireless potentiostat (PalmSens) and PSTracex software (v5.4). Signal results are compared against control, disease-free saliva (also run in triplicate) using Student's t-test. Outliers are identified using Dixon's Q-test, Grubbs' test, and Iglewicz and Hoaglin's test. Results are also plotted using Origin (OriginLab) and MATLAB (MathWorks).

Based on in silico estimates of antigen and antibody homology, and empirical S1(RBD) antibody affinity studies (preliminary studies), no significant cross-reactivity of the capture and detection antibodies with other pathogens is expected. If disease-free saliva samples spiked with influenza or HCoV-NL63 show signals that are significantly higher than control saliva samples indicating potential cross reactivity, additional highly-specific monoclonal anti-S1(RBD) capture and HRP-tagged detection antibodies from another commercial vendor (ProSci, CA) are acquired as alternative solutions to be substituted into the assay.

### Evaluate Saturation, Interference, Inhibition and Sensor Stability

Saliva is a complex matrix, saturated with biomolecules, contaminated with mouth, lung, and gastrointestinal flora and highly variable among individuals. In order to study the potential effects of saturation and inhibition on the performance of the prototype ECIA assay potential variability was first evaluated in performance due to sample collection and inter-individual variability in saliva.

### Effect of Sample Collection Strategy

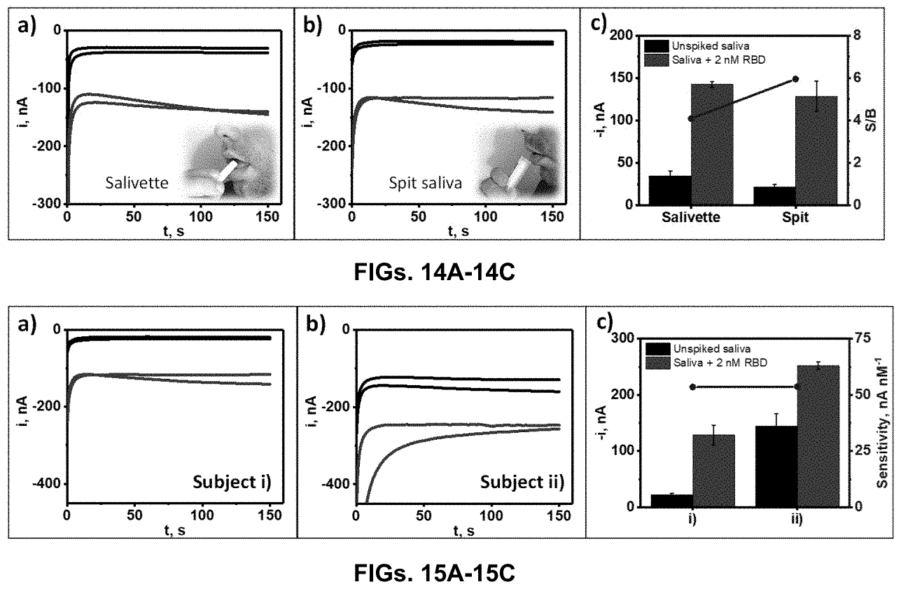

saliva was collected using a Salivette® collector kit (Sarstedt, Nümbrecht, Germany) and also by directly spit into a sterile centrifuge tube. 2 nM of recombinant S1 RBD antigen (CV2—Wuhan strain) was then spiked into the saliva samples and the control (saliva only) was compared to the saliva plus RBD antigen collected with both methods (two replicates each) (FIGS. 14A-14C). Three (3) things were learned: 1) The spiked saliva from both methods showed significantly higher signal than control using either method—indicating strong affinity of the assay antibodies to the target S1 (RBD) antigen; 2) the directly collected saliva (“spit”) showed a stronger signal than salivette collected—indicating potential inhibitors in the salivette and favoring direct collection of saliva; and 3) results met expectations regarding reproducibility.

### Inter-Individual Variability

Individual subject saliva variability was evaluated by directly collecting saliva (spit method) from two individuals with recent negative CV2 RT PCR tests. The same methods as above were then used to spike saliva from each with RBD antigen and evaluated amperometric response from each against control saliva (two replicates) (FIGS. 15A-15C). Two things were learned 1) that while absolute signal varied between individuals, the delta between control and RBD spiked saliva was similar; and 2) that replicate results were reproducible.

The studies have been primarily conducted using purified CV2 RBD antigen spiked into buffer and disease-free saliva. It was studied potential saturation effects and inhibition on the ECIA assay using contrived samples made with quantified, inactivated whole CV2 virus spiked into saliva and buffer.

### Saturation, Inhibition, and Interference

With the objective of avoiding false CV2 negative readouts from the ECIA sensor due to a high-dose Hook effect, the saturation curve of the one-step immunoassay is studied. Three replicates are created, each of a 10-fold serial dilution of saliva samples spiked with quantified, inactivated CV2 ranging from 10²-10¹² viral copies per 10 μL assay reaction, in order to quantify the ECIA amperometric response at each concentration. With these data it is able to define the shape of the dynamic range curve, as well as determine if and when oversaturation of the system occurs.

The direct application of ECIA for the analysis of complex biological fluids, such as untreated saliva matrix, remains a major challenge due to potential electrode surface passivation by the accumulation of biomolecules. This “bio-fouling” commonly involves the adsorption, polymerization or precipitation of non-specific biomolecules that inhibit the antigen-antibody interaction and/or the electron transfer during the oxidation step. For this reason, it is important to characterize the immunosensor performance with inactivated whole virus spiked into both saliva and buffer in order to compare the electrochemical background and the analytical performance of CV2-spiked saliva samples against CV2-spiked buffer.

### Stability

The most important component of the assay from a manufacturing stability perspective is the ECIA sensor. While the plastic and gold are inert and highly stable, once the capture antibody is covalently bonded to the sensor it is potentially vulnerable to degradation. In this experiment batches of sensors are prepared with the anti-S1(RBD) capture antibody attached on the electrode surface and are stored at 4° C. Twice a week the amperometric responses are examined from a batch of three stored sensor chips incubated with saliva samples (unspiked and spiked with a known concentration of inactivated CV2). From these data control charts are created for the ratios of signals corresponding to each CV2 positive/negative test realized with the set of stored chips to ensure that the sensors maintain operational and that performance does not deviate more than 10% over 30 days from fabrication.

Based on the preliminary studies of RBD antigen spiked saliva, it appears that the prototype assay is robust to variability expected from collection method and among individuals, and that the assay delivers reproducible results with little indication of major inhibition or blocking effects from saliva. It does, however, appear that the assay shows signs of saturation around 2 nM of RBD antigen. If whole virus spike studies indicate new inhibition effects, the potential strategies to address these matrix effects and bio-fouling issues in saliva include: 1) Electrode surface chemistry modification with blocking agents (e.g. BSA, whey proteins, animal serum or protein cocktails); 2) Addition of conditioning reagents to saliva samples (surfactants and/or proteins) to optimize conformation for improved antigen-antibody recognition; and 3) saliva pre-treatment to enable target isolation and/or denaturation of interferents, including heat, filtration and centrifugation. Regarding stability, preliminary studies of the similarly designed M. tuberculosis antigen ECIA, indicate no deterioration of performance or sensitivity after 4 weeks at 4° C.; similar performance for the CV2 ECIA is expected.

### Example 6: Lower Limits of Reproducible Detection

Using FDA recommended methods (2-fold dilution series×3 replicates each) the ECIA assay is able to reproducibly detect as few as 10-100 viral copies in a 10 μL sample

Preliminary studies of the prototype ECIA's dynamic range for detection of CV2 RBD antigen spiked into saliva were completed. In brief, anti-S1 (RBD) capture antibody was immobilized on the ECIA sensor surface, then 3 μg/mL of HRP-tagged anti-S1 detection antibody was added to 10 μL of the saliva sample (spiked with RBD antigen at 0, 0.5, 1.0 and 2.0 nM), then placed directly on the sensor and incubated for 20 minutes at room temperature. The sensor was then washed with PB and SDS and then with 0.1M PB (pH 7.4) in order to remove unbound HRP-S1 antibodies. Finally, each chip was dried with compressed air, and the sensor was attached to a potentiostat before a 20 μL drop of TMB/H₂O₂ solution was placed on the sensor covering both working and reference electrodes to initiate the oxidation reaction.

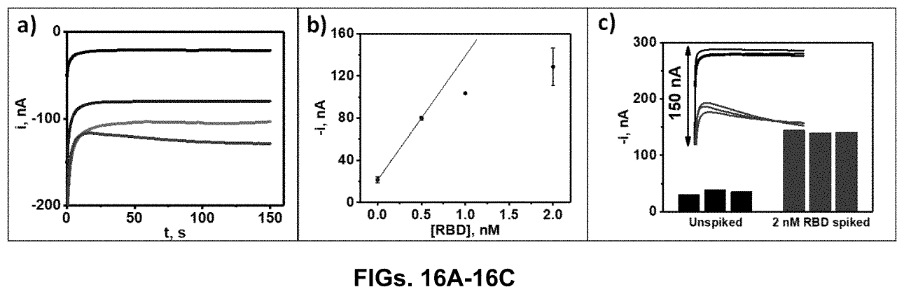

Chronoamperometric measurements were recorded after the application of low potential (˜0.10 V) for 150 s. The observed results (FIGS. 16A-16C) indicate that it was able to discriminate saliva spiked with as little as 500 pM RBD antigen from saliva controls. The calibration data represented in FIG. 16B suggest non-linear response of the immunosensor with >1 nM of antigen, so that the linearity of the system is defined in the low pM range, with a preliminary estimated LoD of ˜117 pM, which corresponds to 38.61 μg RBD in a 10 μL sample droplet. Additionally, the reproducibility evaluation of measurements of saliva samples unspiked and spiked with 2 nM RBD antigen (realized with three different ECIA sensors per concentration) yielded standard deviation values of 4.1 and 2.4 nA, respectively (FIG. 16C), illustrating the reproducibility of the current prototype assay results. Further, these studies are expanded using inactivated whole virus spiked into saliva to establish the assay's LoD for CV2 in saliva.

FDA EUA guidelines are followed for calculating the LoD of the ECIA assay. Based on preliminary studies of the ECIA assay sensitivity based on RBD antigen detection and published clinical studies indicating viral load in clinical samples to range from −10¹-10⁸ viral copies are expected in the 10 μL saliva sample; as well as the dynamic range of the ECIA assay that is established in above saturation studies, the LoD of the assay to fall between 10¹-10³ viral copies per reaction was expected. A 2-fold dilution series (3 replicates each concentration) of between 1000 and 10 viral copies was then developed by spiking quantified whole CV2 virus into saliva and the established LoD with 20 replicates at the LoD (one concentration higher than detectable) were then confirmed.

As per the FDA definition for CV2 antigen assays, the LoD is defined as the lowest concentration at which 19/20 replicates are positive for detection of CV2 virus. Based on preliminary studies of RBD antigen spiked into saliva, indicating the ECIA assay can reproducibly detect 500 pM antigen concentrations, and a LoD of ˜100 viral copies/reaction are expected to be determined. Potential problems may include lack of reproducible results at low viral load and failure to reach expected LoD. While the risk of this is low given the high theoretical and demonstrated sensitivity of the ECIA assay, redox cycling of the signaling molecule to amplify the signal, if needed, could be used.

### Example 7: Confirmation of Results Using RT-PCR

A minimum of 90% positive percent agreement and 98% negative percent agreement with clinical-grade RT PCR test results was achieved.

In order to establish clinical performance of the ECIA assay clinical saliva samples from RT PCR positive COVID-19 patients and RT PCR negative patients under investigation (PUI) was required to be accessed. Matched saliva (raw saliva into a sterile tube) and NP swab samples (stored in viral transport medium) from UCSD outpatients with RT PCR confirmed COVID-19 diagnoses were used for these experiments. The viral load of matched saliva/NP samples were quantified using a standardized multiplex RT PCR reaction targeting 3 different genes on the CV2 genome: N-gene, Orf1ab, and S-gene. Over 80 matched samples have been collected to date from both male and females and a diversity of ethnicities ranging in age from 18-80. These samples were used for the studies described below.

Clinical performance was established according to FDA guidelines. Three replicates of 30 randomly selected, de-identified clinical saliva samples from the biobank of RT PCR positive COVID-19 patients described above, and 30 de-identified saliva samples from RT PCR negative PUIs were tested. All biobank sample results were blinded to the testing team. The ECIA was run using workflow described above. Amperometric results for replicates were used to calculate a mean and SD for each sample and were compared to a control threshold value established using three independent disease-free saliva samples. All samples significantly above the threshold were considered “positive”, while those below the control threshold value were recorded as “negative”. The reference RT PCR results were then unblinded and the positive percent and negative percent agreement are determine based on concordance or discordance with reference RT PCR results from the NP swab samples. Viral load was also estimated in each sample based on saturation curves obtained as described in above Examples 3 and 4 above and is then compared to estimates of viral load in the saliva and NP samples as determined by RT PCR.

The US Food and Drug Administration and the World Health Organization require a minimum positive percent agreement with RT PCR reference testing of 80% and a negative percent agreement of 97%. Based on the preliminary studies, the ECIA assay exceeds these performance requirements with clinical samples. If the positive percent agreement threshold had not been met, it could have been due to saliva matrix affects which can include a wide variety of inhibitors, or saturation effects due to mismatches of antibody density on the chip and high viral load. In this case methods outlined in above Example 3 could have been used to mitigate these problems. In the unlikely event low sensitivity issues with saliva could be resolved, the same approach as that used by only two FDA approved CV2 antigen assays, which was to switch to a standardized NP swab sample in viral transport medium to simplify the sample matrix, could also have been taken.

### Example 8: Initial Clinical Saliva Sample Results

### Studies Conducted Using Fresh Undiluted Human Saliva

These studies provided evaluation of the sensor performance in the detection of RBD in spiked saliva samples. Spit saliva samples used were collected from two individuals tested negative for COVID-19 on Jul. 11, 2020 and Jun. 28, 2020, respectively. Saliva was collected using Sarstedt Cortisol Salivette®.

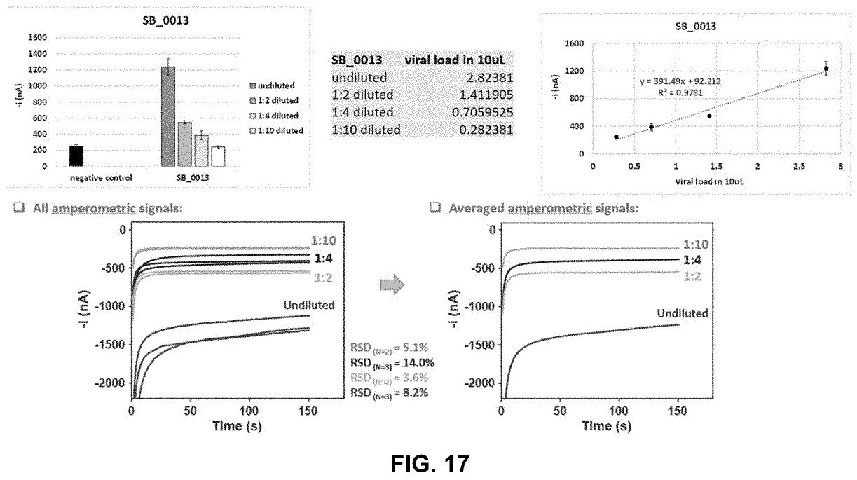

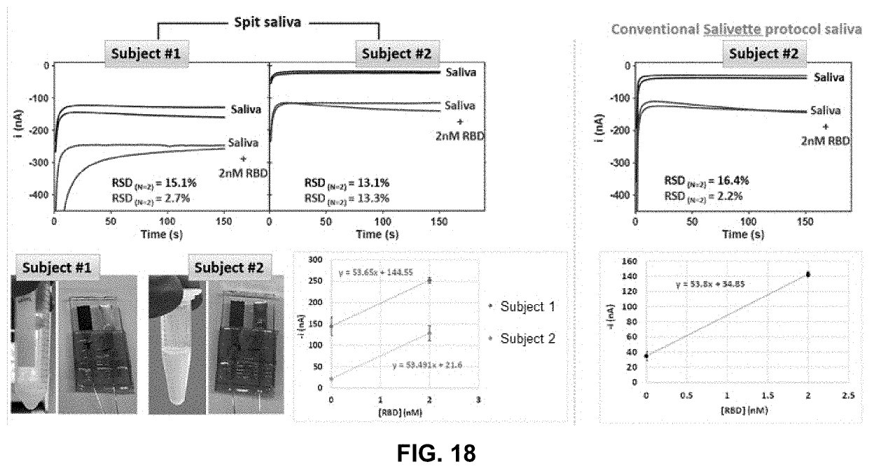

The same slope was obtained for both spit samples tested. The difference in the currents recorded may have been due to the different matrices: one sample presented higher amount of cells/biological material than the other, providing higher background signals, but the same slope obtained for both spit saliva and salivette-collected saliva. Spit saliva provided a bit lower current signals compared to use of the salivette protocol as previously obtained with previous HRP-Ab. (See FIGS. 17-18.)

### COVID-19 RBD Antigen Calibration in Undiluted Spit Saliva

Calibration was performed using RBD-spiked spit saliva with a COVID-19 negative saliva sample as reference matrix with lower background signals.

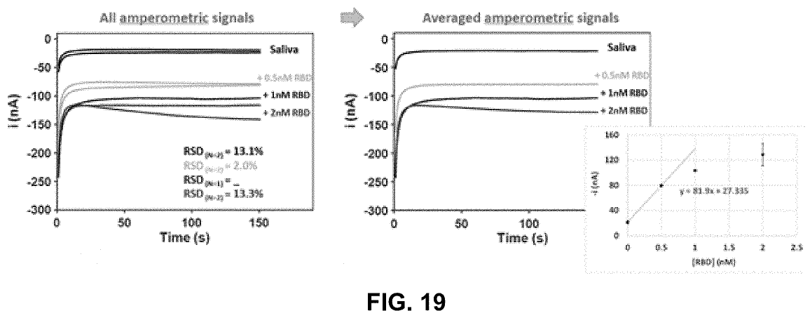

Satisfactory detection of 500 pM RBD was achieved. Sensors were saturated over this concentration. It would be desirable to repeat calibration in saliva to check linear concentration range for this COVID-19 antigen. (See FIG. 19.)

### Example 9: Clinical Serum and Urine Sample Results

In total 33 serum and 10 urine samples were evaluated from three independent cohorts of patients considered at risk for active TB or drug resistant disease (see Table 5). Assay results from TB positive patients indicated that circulating CFP-10 antigens were detected in ALL serum (n=19) and urine (n=3) samples from positive TB patients who were untreated at time of serum collection or had <1 week of treatment. Conversely, serum (n=7) and urine (n=6) samples collected from patients at risk for TB, but who were TB negative by culture, AFB and/or GeneXpert had CFP-10 signals that were not significantly different from negative controls. TB patients with a record of week treatment produced results both above and below the negative control threshold, with values mostly inversely correlated with days of treatment. The resulting preliminary sensitivity and specificity of TB detection among participants with <1 week of treatment compared to TB negative was therefore 100% (95% CI: 81%-100%) and 100% (95% CI: 72%-100%), respectively.

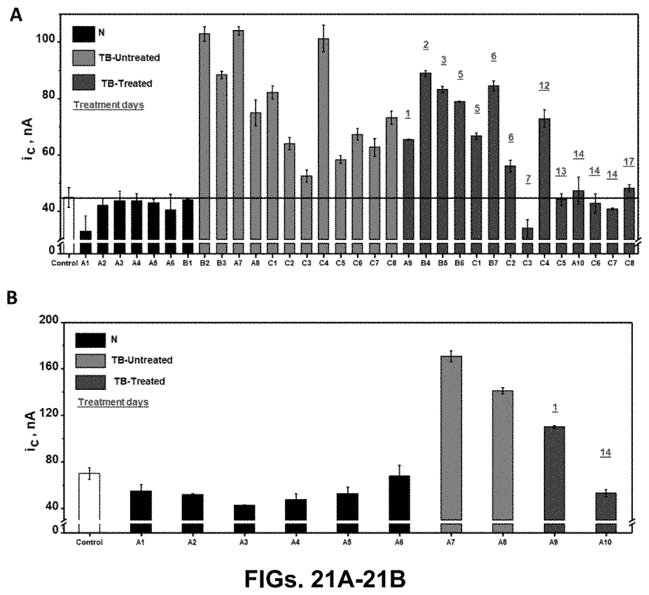

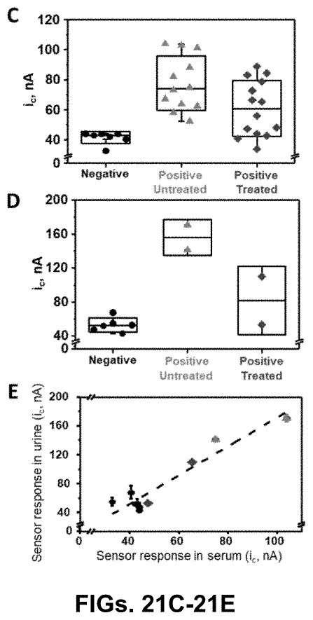

Table 5 shows amperometric response and estimated concentration of Mtb CFP-10 antigen in clinical serum and urine samples collected from microbiologically confirmed TB positive and TB negative patients using the disclosed assay and test strip. CFP-10 concentration derived from concentration curves established for the assay for serum and urine sample matrices independently (FIGS. 5A and 5C; see also FIGS. 21A-21E).

TABLE 5
Clinical Testing of Serum and Urine Samples

| | | | |
|---|---|---|---|
| | | Amperometric | CFP-10 |
| Patient | Days on | response | Concentration |
| ID# | treatment | (−I, nA) | (nM) |

Serum Samples

| | | | | |
|---|---|---|---|---|
| TB | A1 | n/a | 32.8 ± 5.5 | ND |
| Negative | A2 | n/a | 42.2 ± 2.3 | ND |
| | A3 | n/a | 43.8 ± 3.3 | ND |
| | A4 | n/a | 43.8 ± 2.4 | ND |
| | A5 | n/a | 43 ± 1.2 | ND |
| | A6 | n/a | 40.4 ± 5.5 | ND |
| | B1 | n/a | 44.1 ± 0.3 | ND |
| TB | A7 | 0 | 103.9 ± 1.5 | 5.9 ± 0.4 |
| Positive | A8 | 0 | 74.9 ± 4.5 | 3.8 ± 0.9 |
| | A9 | 1 | 65.4 ± 0.1 | 1.8 ± 0.3 |
| | A10 | 14 | 47.3 ± 4.9 | ND |
| | B2 | 0 | 102.9 ± 2.6 | 5.8 ± 0.6 |
| | B3 | 0 | 88.3 ± 1.3 | 4.5 ± 0.3 |
| | B4 | 2 | 88.9 ± 1 | 4.5 ± 0.8 |
| | B5 | 3 | 83.1 ± 1.3 | 4.0 ± 0.3 |
| | B6 | 4 | 78.8 ± 0.2 | 3.6 ± 0.2 |
| | B7 | 6 | 84.4 ± 1.8 | 4.1 ± 0.4 |
| | C1 | 0 | 82 ± 2.3 | 3.9 ± 0.5 |
| | C1 | 5 | 66.6 ± 1.2 | 2.4 ± 0.9 |
| | C2 | 0 | 63.9 ± 2.2 | 2.2 ± 0.5 |
| | C2 | 6 | 56 ± 2.1 | 1.5 ± 0.5 |
| | C3 | 0 | 52.5 ± 2.2 | 1.1 ± 0.5 |
| | C3 | 7 | 33.9 ± 3.1 | ND |
| | C4 | 0 | 101.2± 4.8 | 5.7 ± 0.7 |
| | C4 | 12 | 72.8 ± 3.2 | 3.0 ± 0.9 |
| | C5 | 0 | 58.2 ± 1.3 | 1.7 ± 0.3 |
| | C5 | 13 | 44.2 ± 2 | ND |
| | C6 | 0 | 67.3 ± 2 | 2.5 ± 0.5 |
| | C6 | 14 | 42.8 ± 3.4 | ND |
| | C7 | 0 | 62.6 ± 3.1 | 2.1 ± 0.7 |
| | C7 | 14 | 40.8 ± 0.4 | ND |
| | C8 | 0 | 73.2 ± 2.3 | 3.1 ± 0.5 |
| | C8 | 17 | 48.2 ± 1.2 | 0.73 ± 0.01 |

Urine Samples

| | | | | |
|---|---|---|---|---|
| TB | A1 | n/a | 54.8 ± 5.5 | ND |
| Negative | A2 | n/a | 52 ± 0.8 | ND |
| | A3 | n/a | 43 ± 0.2 | ND |
| | A4 | n/a | 47.7 ± 5.1 | ND |
| | A5 | n/a | 52.8 ± 5.7 | ND |
| | A6 | n/a | 67.9 ± 9 | ND |
| TB | A7 | 0 | 170.6 ± 4.7 | 8.8 ± 0.9 |
| Positive | A8 | 0 | 141.1 ± 2.6 | 6.3 ± 0.5 |
| | A9 | 1 | 110 ± 1.1 | 3.6 ± 0.2 |
| | A10 | 14 | 53.1 ± 3.1 | ND |

Analytical Performance using Clinical Samples

Amperometric responses for all clinical serum and urine samples evaluated (in triplicate) are presented in FIGS. 21A-21B. Based on the assumption that patients that had been treated for TB for more than a few days could have reduced CFP-10 signal, microbiologically confirmed TB positive patients were stratified into those that had received zero days of treatment at time of sample collection (untreated), from those that had received between 1-17 days of treatment (treated). Mean signals from clinical serum samples from TB negative, TB positive (untreated), and TB positive (treated) individuals, were 41.4 nA, 77.6 nA and 60.9 nA respectively, see FIG. 21C. Mean amperometric signals from clinical urine samples from TB negative, TB positive (untreated) and TB positive (treated) patients, were 53.0 nA, 155.8 nA and 81.6 nA, respectively, see FIG. 21D. Amperometric responses from matched serum and urine samples from the same patient, regardless of treatment days, were highly correlated Pearson's r=0.95, p<0.01 (FIG. 21E). Using concentration curves derived independently for serum and urine (FIGS. 5A and 5C), we estimated CFP-10 concentrations in the clinical serum samples ranged from 1.1 nM to 5.9 nM in TB positive patients with <7 days of treatment, while patients with days of treatment had mostly undetectable levels of CFP-10 (Table 5). CFP10 concentrations in urine appeared to be 1.5-2 times higher than in matched serum samples from the same patient.

### Treatment Response

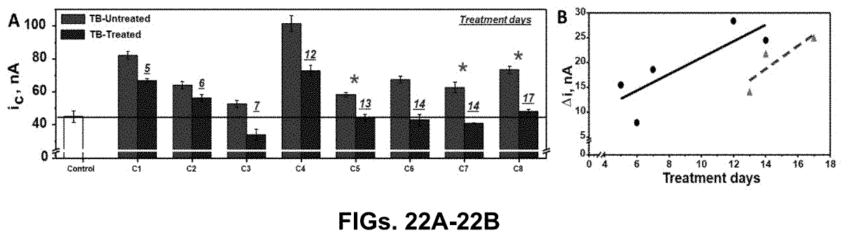

We evaluated pairs of serial samples from eight patients from study Cohort C to examine treatment response in drug resistant TB patients. All eight patients were sputum smear and culture positive prior to treatment initiation. Follow-up samples were collected between 1-3 weeks after treatment initiation. Follow up samples from five of the patients were smear and culture negative at follow up while three patients were smear negative, but still culture positive at follow up. The BETA assay signals from patients at baseline (untreated) and follow-up (treated) can be seen in FIG. 22A. The difference between the amperometric signal at follow and before treatment initiation (in triplicate) were plotted against number of treatment days (FIG. 22B), with separate slopes for patients that became culture negative (n=5) and those that did not culture convert (n=3). Controlling for follow-up culture status, days on treatment was significantly associated with decrease in CFP-10 signal by linear regression (p=0.023, R2=0.68), suggesting CFP-10 reduction could be correlated with treatment response.

### Example 10: Conclusions

The disclosed bioelectronic immunoassay is useful for detecting clinically relevant concentrations of CFP-10 and/or other antigens directly from unprocessed small-volume clinical serum or urine samples, indicating its potential as a point-of-care diagnostic for active TB disease and/or other infectious diseases. Using one embodiment of the disclosed assay, we detected CFP-10 antigen in concentrations ranging from 1.1 nM to 5.9 nM in serum and 3.6 nM to 8.8 nM in urine in all culture positive TB patients with less than one week of treatment. Previous immunoassays for detecting CFP-10 have been limited to detection of antigens in highly processed clinical samples, contrived laboratory samples, sputum samples, or culture filtrate samples. Complex sample preparation protocols and costly instrumentation, however, preclude the utility of these technologies as point-of-care diagnostic solutions.

In addition, assay results of paired serum samples collected from drug resistant TB patients before and after treatment initiation suggested a correlation between decrease in CFP-10 antigen signal and days on treatment, after controlling for final culture status. Although only a limited number of patients were evaluated, this data suggests that the BETA assay could not only be used as a point-of-care diagnostic to rapidly detect active TB disease, but it could also be used to evaluate treatment response, allowing clinicians timely treatment regimen modification, in contrast to the reference standard, which is based on slow growing cultures that can take several weeks.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Azzi L, et al. Saliva is a reliable tool to detect SARS-CoV-2. The Journal of infection. 2020, 81:e45-e50.
2. Broger, T. et al. Diagnostic accuracy of three urine lipoarabinomannan tuberculosis assays in HIV-negative outpatients. The Journal of clinical investigation, 130:5756-5764 (2020).
3. Bussi, C. et al. Mycobacterium tuberculosis infection of host cells in space and time. FEMS microbiology reviews 43:341-361 (2019).
4. COVID-19 Dashboard by the Center for Systems Science and Engineering (CSSE) at Johns Hopkins University (JHU), accessed Aug. 12, 2020.
5. Esposito, S., et al. Tuberculosis in children. Mediterranean journal of hematology and infectious diseases 5:e2013064 (2013).
6. Fan, J. et al. Rapid diagnosis of new and relapse tuberculosis by quantification of a circulating antigen in HIV-infected adults in the Greater Houston metropolitan area. BMC medicine 15:188 (2017).
7. FIND. Expression of interest: Driving equitable access to fit-for-purpose antigen-detecting rapid diagnostic tests for COVID-19, accessed Aug. 12, 2020.
8. Flores, L. L. et al. Systematic review and meta-analysis of antigen detection tests for the diagnosis of tuberculosis. Clinical and vaccine immunology: CVI 18:1616-1627 (2011).
9. Getahun, H., Harrington, M., O'Brien, R. & Nunn, P. Diagnosis of smear-negative pulmonary tuberculosis in people with HIV infection or AIDS in resource-constrained settings: informing urgent policy changes. Lancet 369:2042-2049 (2007).
10. Global tuberculosis report 2020. World Health Organization, Geneva (2020).
11. Kadir, M. K. et al. Development of an electrochemical immunosensor for fumonisins detection in foods. Toxins 2:382-398 (2010).
12. Kik, S. V., et al. Tuberculosis diagnostics: which target product profiles should be prioritised? The European respiratory journal 44:537-540 (2014).
13. Kim, J. et al. Clinical immunosensing of tuberculosis CFP-10 antigen in urine using interferometric optical fiber array. Sensors and Actuators B: Chemical 216:184-191 (2015).
14. Kim, J. et al. Plastic-Chip-Based Magnetophoretic Immunoassay for Point-of-Care Diagnosis of Tuberculosis. ACS applied materials & interfaces 8:23489-23497 (2016).
15. Lee G Y, et al. Chronoamperometry-Based Redox Cycling for Application to Immunoassays. ACS Sens. 2018; 3(1):106-12.
16. Li, J., et al. Click DNA cycling in combination with gold nanoparticles loaded with quadruplex DNA motifs enable sensitive electrochemical quantitation of the tuberculosis-associated biomarker CFP-10 in sputum. Microchimica Acta 186:662 (2019).
17. Liu, C. et al. Quantification of circulating Mycobacterium tuberculosis antigen peptides allows rapid diagnosis of active disease and treatment monitoring. Proc Natl Acad Sci USA 114:3969-3974 (2017).
18. Mohd Azmi, U. Z. et al. Sandwich Electrochemical Immunosensor for Early Detection of Tuberculosis Based on Graphene/Polyaniline-Modified Screen-Printed Gold Electrode. Sensors 18:3926 (2018).
19. Nkereuwem, E. et al. Comparing accuracy of lipoarabinomannan urine tests for diagnosis of pulmonary tuberculosis in children from four African countries: a cross-sectional study. The Lancet Infectious Diseases, 21:376-384 (2020).
20. Ou X, et al. Characterization of spike glycoprotein of SARS-CoV-2 on virus entry and its immune cross-reactivity with SARS-CoV. Nature communications. 2020, 11:1620.
21. Pai, M. et al. Tuberculosis Diagnostics in 2015: Landscape, Priorities, Needs, and Prospects. The Journal of infectious diseases 211:S21-S28 (2015).
22. Pai, M. et al. Tuberculosis. Nature reviews. Disease primers 2:16076 (2016).
23. Péterfi, Z. et al. Comparison of Blocking Agents for an Elisa for Lps. Journal of Immunoassay 21:341-354 (2000).
24. Ponnudurai, N., et al. New TB Tools Need to be Affordable in the Private Sector: The Case Study of Xpert MTB/RIF. Journal of epidemiology and global health 8:103-105 (2018).
25. Puri, L., et al. Xpert MTB/RIF for tuberculosis testing: access and price in highly privatised health markets. The Lancet. Global health 4:e94-95, doi:10.1016/52214-109X(15)00269-7 (2016).
26. Ruiz-Valdepeñas Montiel, V. et al. Delayed Sensor Activation Based on Transient Coatings: Biofouling Protection in Complex Biofluids. J Am Chem Soc 140:14050-14053 (2018).
27. Sarro, Y. D. et al. Simultaneous diagnosis of tuberculous and non-tuberculous mycobacterial diseases: Time for a better patient management. Clin Microbiol Infect Dis 3:10.15761/CMID.1000144 (2018).
28. Shang J, et al. Structural basis of receptor recognition by SARS-CoV-2. Nature. 2020, 581:221-224.
29. SinoBiological: SARS-CoV-2 antibodies and antibody guide. Available from: <https://www.sinobiological.com/research/virus/sars-cov-2-antibody>, accessed Jun. 23, 2021.
30. Tang, Z. et al. Sensitive immunoassays of nitrated fibrinogen in human biofluids. Talanta 81:1662-1669 (2010).
31. The Lancet Infectious, D. Tuberculosis and malaria in the age of COVID-19. The Lancet Infectious Diseases 21, 1, doi:https://doi.org/10.1016/S1473-3099(20)30946-4 (2021).
32. To K K, et al. Consistent detection of 2019 novel coronavirus in saliva. Clinical infectious diseases: an official publication of the Infectious Diseases Society of America. 2020. 7108139
33. To K K, et al. Temporal profiles of viral load in posterior oropharyngeal saliva samples and serum antibody responses during infection by SARS-CoV-2: an observational cohort study. The Lancet Infectious diseases. 2020; 20(5):565-574.
34. Tufa, L. T. et al. Electrochemical immunosensor using nanotriplex of graphene quantum dots, Fe₃O₄, and Ag nanoparticles for tuberculosis. Electrochimica Acta 290:369-377 (2018).
35. USFDA. Policy for Coronavirus Disease-2019 Tests During the Public Health Emergency, accessed Jun. 23, 2021.
36. van Pinxteren, L. A., et al. Diagnosis of tuberculosis based on the two specific antigens ESAT-6 and CFP10. Clinical and diagnostic laboratory immunology 7:155-160 (2000).
37. Vargas, E. et al. Enzymatic/Immunoassay Dual-Biomarker Sensing Chip: Towards Decentralized Insulin/Glucose Detection. Angew Chem Int Ed Engl, 58:6376-6379 (2019).
38. World Health Organization, The End TB Strategy: Global strategy and targets for tuberculosis prevention, care and control after 2015, (2014).
39. Wyllie A L, et al. Saliva or nasopharyngeal swab specimens for detection of SARS-CoV-2, N Engl J Med, 2020, 383:1283-1286.
40. Yáñez-Sedeño, P., et al. Multiplexed Electrochemical Immunosensors for Clinical Biomarkers. Sensors 17:965 (2017).
41. Zheng M, et al. Novel antibody epitopes dominate the antigenicity of spike glycoprotein in SARS-CoV-2 compared to SARS-CoV. Cellular & molecular immunology. 2020, 17:536-538.

1. A test strip for detecting a disease or monitoring treatment of a disease in a subject, the test strip comprising a working electrode and a combination reference and counter electrode, wherein the working electrode and the combination reference and counter electrode comprise a substrate coated with a metal, and wherein the metal is bonded to a capture antibody for an antigen associated with the disease.

2. The test strip of claim 1, wherein the metal comprises a thin film, and wherein the metal comprises chromium, gold, or a combination thereof.

3-5. (canceled)

6. The test strip of claim 1, wherein the capture antibody is coupled to the metal of the working electrode and the combination reference and counter electrode using carboxyl-amine coupling, thiolate self-assembled monolayer, 4-carboxymethylaniline conjugation, or any combination thereof.

7. The test strip of claim 1, wherein the substrate comprises a plastic material or glass, and wherein the plastic material comprises polyethylene terephthalate glycol (PETG), polybutylene terephthalate (PBD), polyethylene terephthalate (PET), ethylene vinyl acetate (EVA), acrylonitrile butadiene styrene (ABS), polytetrafluoroethylene (PTFE), polyamide, polyetheretherketone (PEEK), polycarbonate, polyethylene terephthalate polyester (PETP), high density polyethylene (HDPE), low density polyethylene (LDPE), linear low density polyethylene (LLDPE), polymethylpentene (PMP), poly(p-phenylene oxide) (PPO), polypropylene, high impact polystyrene (HIPS), polyvinyl chloride (PVC), styrene acrylonitrile (SAN), acrylonitrile styrene acrylate, polyvinyl alcohol (PVOH), or any combination thereof.

8. (canceled)

9. The test strip of claim 1, wherein the disease comprises tuberculosis, COVID-19, HIV, coccidiomycosis, a disease caused by a non-tuberculosis Mycobacterium species, hepatitis A, hepatitis B, hepatitis C, hepatitis D, or hepatitis E.

10. The test strip of claim 9, wherein the test strip is not cross reactive with an antigen from a disease not being detected or monitored.

11. The test strip of claim 9, wherein the disease is tuberculosis and the capture antibody comprises an anti-CFP-10 antibody, an anti-ESAT-6 antibody, an anti-MPT64 antibody, an anti-Ag85B antibody, an anti-LAM antibody, or a combination thereof.

12. The test strip of claim 9, wherein the disease is COVID-19 and the capture antibody comprises an anti-receptor binding domain (anti-RBD) antibody, an anti-S1 spike subunit antibody, an anti-nucleocapsid protein antibody, or a combination thereof.

13. The test strip of claim 1, wherein the subject is a mammal and/or human.

14. (canceled)

15. The test strip of claim 1, wherein the capture antibody has a capture efficiency of at least 50%.

16. (canceled)

17. The test strip of claim 1, wherein the test strip is disposable.

18. A method for detecting a disease in a subject, the method comprising at least the following steps:
(a) incubating the test strip of claim 1 with a biological sample from the subject;
(b) rinsing the test strip to remove unbound material;
(c) incubating the test strip with a detection antibody specific to at least one disease-related antigen, the detection antibody comprising at least one tag;
(d) rinsing the test strip to remove unbound detection antibody;
(e) applying a current to the test strip; and
(f) detecting a signal from the at least one tag.

19. The method of claim 18, wherein the disease comprises tuberculosis, COVID-19, HIV, coccidiomycosis, a disease caused by a non-tuberculosis Mycobacterium species, hepatitis A, hepatitis B, hepatitis C, hepatitis D, or hepatitis E.

20. The method of claim 18, wherein the biological sample comprises blood, serum, saliva, or urine that directly incubate with the test strip.

21-25. (canceled)

26. The method of claim 18, wherein the at least one tag comprises horseradish peroxidase (HRP), an enzyme label, or any combination thereof.

27. (canceled)

28. The method of claim 26, wherein the at least one tag is HRP and the method further comprises following step (d) but prior to step (e), contacting the test strip with a solution of 3,3′,5,5′-tetramethylbenzidine (TMB) and H₂O₂.

29. The method of claim 18, wherein the signal from the at least one tag comprises an amperometric signal, wherein a magnitude of the amperometric signal corresponds to a concentration of the antigen in the biological sample, and/or directly proportional to an amount of HRP-tagged antibody bound to the test strip.

30-35. (canceled)

36. The method of claim 29, further comprising redox cycling to amplify the signal.

37-40. (canceled)

41. The method of claim 18 wherein the method further comprises quantifying an amount of the at least one disease-related antigen in the biological sample.

42. A method for monitoring treatment of an infectious disease in a subject, the method comprising:
(g) performing the method of claim 18 in a subject having the infectious disease a first time and obtaining a first antigen quantity;
(h) treating the infectious disease; and
(i) performing the method of claim 18 in the subject a second time and obtaining a second antigen quantity;
wherein successful treatment of the infectious disease is indicated by a lower second antigen quantity relative to the first antigen quantity.
